(19) United States
(12) Patent Application Publication
(10) Pub. No.: US 2024/0018499 A1
(43) Pub. Date: Jan. 18, 2024

(54) ACE2 Fusion Proteins and Uses Thereof

(71) Applicant: Formycon AG, Martinsried/Planegg (DE)

(72) Inventors: Alwin Reiter, München (DE); Carsten Brockmeyer, Marzling (DE); Florian Wolschin, München (DE)

(21) Appl. No.: 18/033,793

(22) PCT Filed: Oct. 29, 2021
(86) PCT No.: PCT/EP2021/080130

§ 371 Date: Apr. 25, 2023

(30) Foreign Application Priority Data
Oct. 29, 2020 (EP) 20204774.2
Nov. 27, 2020 (EP) 20210297.6
Mar. 24, 2021 (EP) 21164684.9
Apr. 26, 2021 (EP) 21170519.9

(51) Int. Cl. C12N 9/48; A61K 38/48; A61P 31/14

(52) U.S. Cl. CPC C12N 9/485; C12Y 304/17023; A61K 38/4813; A61P 31/14; C07K 2319/30; A61K 38/00

(57) ABSTRACT

The present invention relates to fusion proteins of ACE2 with IgG Fc and the medical use of these fusion proteins, in particular in the prevention or treatment of infections with coronaviruses such as SARS-CoV-2.

### FIELD OF THE INVENTION

The present invention relates to fusion proteins of ACE2 with IgG Fc and the medical use of these fusion proteins, in particular in the prevention or treatment of infections with coronaviruses such as SARS-CoV-2.

### BACKGROUND OF THE INVENTION

ACE2 (angiotensin converting enzyme 2) is a key metalloprotease of the renin-angiotensin system with a catalytic zinc atom in the centre (Donoghue et al. (2002) Circ. Res. 87: e1-e9). Full-length ACE2 consists of an N-terminal extracellular peptidase domain, a collectrin-like domain, a single transmembrane helix and a short intracellular segment. It acts to cleave Angiotensin II to produce Angiotensin (1-7) and Angiotensin Ito produce Angiotensin (1-9) which is then processed by other enzymes to become Angiotensin (1-7). ACE2 acts to lower the blood pressure and counters the activity of ACE in order to maintain a balance in the Ras/MAS system. Accordingly, it is a promising target for the treatment of cardiovascular diseases.

Recently, ACE2 has gained much attention as a receptor for coronaviruses and in particular the novel coronavirus SARS-CoV-2. SARS-CoV-2 is a coronavirus which was first discovered in December 2019 in Wuhan, China, but spread rapidly all over the world, leading to a world-wide pandemic. On 22 May 2020, the Johns Hopkins University counted more than 5 millions of confirmed infections worldwide, causing the death of more than 330,000 people. The pandemic led to a lock-down in many countries with very significant economic and social effects.

It was shown that ACE2 functions as a receptor for SARS-CoV (Li et al. (2003) Nature 426: 450-454; Prakabaran et al. (2004) Biochem. Biophys. Res. Comm. 314: 235-241) and for SARS-CoV-2 (Yan et al. (2020) Science 367: 1444-1485). Further, entry of SARS-CoV-2 into the respiratory cells depends on ACE2 and the serine protease TMPRSS2 (Hoffmann et al. (2020) Cell 181: 1-10).

In view of the important role of ACE2 for virus entry into the cell, it was proposed to use soluble ACE2 for blocking SARS binding to the cell (WO 2005/032487; WO 2006/122819). The same approach was also suggested for the treatment of infections with SARS-CoV-2 (Kruse (2020) F1000Res. 9:72). Clinical trials with a soluble form of ACE2 in the treatment of infections with SARS-CoV-2 have been initiated by the company Apeiron (Pharmazeutische Zeitung, 10 Apr. 2020) and the first results showed that one patient with severe Covid-19 caused by SARS-CoV-2 recovered fast upon treatment with soluble ACE2 (Zoufaly et al. (2020) The Lancet Respiratory Medicine 8: 1154-1158, available at https://doi.org/10.1016/S2213-2600(20)30418-5).

However, isolated receptor domains are typically characterized by a low stability and plasma half-life. For the soluble form of ACE2 a dose-dependent terminal half-life of 10 hours was shown (Haschke et al. (2013) Clin. Pharmacokinet. 52: 783-792). In view of these results, it was decided to administer the soluble form of ACE2 as twice-daily infusion in a later study (Khan et al. (2017) Critical Care 21: 234). However, an administration of more than one time per day is inconvenient both for the patient and the medical personnel.

A fusion protein of ACE2 consisting of the extracellular domain of either enzymatically active or enzymatically inactive ACE2 linked to the Fc domain of human IgG1 was constructed and tested. It was shown that both constructs potently neutralized both SARS-CoV and SARS-CoV-2 and inhibited S (Spike) protein-mediated fusion (Lei et al. (2020) Nature Communications 11: 2070). Further, a fusion protein called COVIDTRAP™ or STI-4398 was developed for clinical testing by the company Sorrento Therapeutics (see https://www.globenewswire.com/news-release/2020/03/20/2003957/0/en/SORRENTO-DEVELOPS-STI-4398-COVIDTRAP-PROTEIN-FOR-POTENTIAL-PREVENTION-AND-TREATMENT-OF-SARS-COV-2-CORONAVIRUS-DISEASE-COVID-19.html). Liu et al. (2020) Int. J. Biol. Macromol. 165: 1626-1633, available at https://www.biorxiv.org/content/10.1101/2020.08.13.248351v1.full.pdf, describe fusion proteins of wild-type ACE2 and nine ACE2 mutants affecting catalytic activity of ACE2 with the Fc region of human IgG1. However, the interaction of the Fc domain of human IgG1 with Fc gamma receptors on immune cells may enhance the virus infection (Perlman and Dandekar (2005) Nat. Rev. Immunol. 5(12): 917-927; Chen et al. (2020) Current Tropical Medicine Reports 3:1-4).

Tada et al. (2020), available at https://www.biorxiv.org/content/10.1101/2020.09.16.300319v1.full, disclose an “ACE2 microbody” in which the extracellular domain of catalytically inactive ACE2 is fused to Fc domain 3 of the immunoglobulin heavy chain.

Svilenov et al. (2020), available at https://www.researchsquare.com/article/rs-459941/v1 and PCT/EP2021/063692 describe ACE2 fusion proteins with the Fc domain of IgG1 and IgG4.

European patent application EP21188832.6 discloses ACE2 fusion proteins with the Fc domain of IgM and IgG2.

WO 2021/170113, WO 2021/183404, US 2021/0284716 and WO 2021/189772 also disclose fusion proteins of ACE2 with the Fc part of different immunoglobulins.

Zhang et al. (2021) Cell Discovery 7: 65 describe that a fusion protein of the extracellular domain of ACE2 with the Fc part of IgG1 protected mice from SARS-CoV2 infection, as determined by reduced viral replication, weight loss, histological changes and inflammation in the lungs. Additionally, it is shown that unlike antibodies targeting the receptor-binding domain of SARS-CoV2 treatment with the fusion protein does not induce the development of escape mutants.

Hence, there is still a need for agents which can be used for the treatment and/or prevention of infections with coronaviruses, in particular with SARS-CoV-2.

### SUMMARY OF THE INVENTION

The present invention provides a fusion protein comprising a first part comprising a fragment of human ACE2 or a variant of said fragment, said human ACE2 having the amino acid sequence according to SEQ ID No. 1, and a second part comprising a variant of the Fc portion of human IgG1, said variant having the amino acid sequence according to SEQ ID No. 19.

In one embodiment the first part and the second part are linked by the amino acid sequence according to SEQ ID No. 15.

The fragment of human ACE2 may consist of a protein with the amino acid sequence according to SEQ ID No. 2 or may be the extracellular domain of ACE2 consisting of a protein with the amino acid sequence according to SEQ ID No. 3.

In one embodiment the variant of the human ACE2 fragment is an enzymatically inactive variant of human ACE2.

The enzymatically inactive variant of human ACE2 may comprise a H374N and a H378N mutation, the numbering referring to SEQ ID No. 1.or a mutation at position 273, preferably a R273A mutation, the numbering referring to SEQ ID No. 1.

In one embodiment, the enzymatically inactive variant of human ACE2 consists of a protein having the amino acid sequence according to SEQ ID No. 91 or 92.

In one embodiment, the enzymatically inactive variant of human ACE2 consists of a protein having the amino acid sequence according to SEQ ID No. 24 or 25.

In one embodiment, the variant of the human ACE2 fragment comprises a S645C mutation, the numbering referring to SEQ ID No. 1. In particular, the variant of the human ACE2 fragment may consist of a protein having the amino acid sequence according to SEQ ID No. 28 or 29.

In one embodiment, the variant of the human ACE2 fragment further comprises a H374N and a H378N mutation, the numbering referring to SEQ ID No. 1. In particular, the variant of the human ACE2 fragment may consist of a protein having the amino acid sequence according to SEQ ID No. 32 or 33.

In one embodiment, the variant of the human ACE2 fragment further comprises a mutation at position 273, preferably a R273A mutation, the numbering referring to SEQ ID No. 1. In particular, the variant of the human ACE2 fragment may consist of a protein having the amino acid sequence according to SEQ ID No. 36 or 37.

The present invention further provides a fusion protein comprising:
(a) a first part comprising a variant of a fragment of human ACE2, said human ACE2 having the amino acid sequence according to SEQ ID No. 1, wherein the variant of the human ACE2 fragment comprises a S645C mutation, the numbering referring to SEQ ID No. 1, and
(b) a second part comprising the Fc portion of human IgG or a variant of the Fc portion of human IgG.

In one embodiment, the variant of a fragment of human ACE2 consists of a protein having the amino acid sequence according to SEQ ID No. 28 or 29.

In one embodiment, the variant of a fragment of human ACE2 further comprises a H374N and a H378N mutation, the numbering referring to SEQ ID No. 1. In particular, the variant of the human ACE2 fragment may consist of a protein having the amino acid sequence according to SEQ ID No. 32 or 33.

In one embodiment, the variant of a fragment of human ACE2 further comprises a mutation at position 273, preferably a R273A mutation, the numbering referring to SEQ ID No. 1. In particular, the variant of the human ACE2 fragment may consist of a protein having the amino acid sequence according to SEQ ID No. 36 or 37.

In one embodiment, the second part comprises the Fc part of human IgG1 or IgG4 or a variant of the Fc part of human IgG1 or IgG4.

In one embodiment, the second part comprises the Fc part of human IgG1 according to SEQ ID No. 16, preferably the first part and the second part are linked by the amino acid sequence according to SEQ ID No. 15.

In another embodiment, the second part comprises the Fc part of human IgG4 according to SEQ ID No. 5, preferably the first part and the second part are linked by the amino acid sequence according to SEQ ID No. 4.

In another embodiment, the second part comprises a variant of the Fc portion of human IgG4 having the sequence according to SEQ ID No. 44, preferably the first part and the second part are linked by the amino acid sequence according to SEQ ID No. 4.

In another embodiment, the second part comprises a variant of the Fc portion of human IgG1 having the sequence according to SEQ ID No. 59 or a variant of the Fc portion of human IgG4 having the sequence according to SEQ ID No. 60.

In one embodiment, the second part comprises a variant of the Fc portion of human IgG1 having the sequence according to SEQ ID No. 59 and the first part and the second part are linked by the amino acid sequence according to SEQ ID No. 15.

In another embodiment, the second part comprises a variant of the Fc portion of human IgG4 having the sequence according to SEQ ID No. 60 and the first part and the second part are linked by the amino acid sequence according to SEQ ID No. 4.

The present invention further provides a fusion protein comprising a first part comprising a fragment of human ACE2 or a variant of said fragment, said human ACE2 having the amino acid sequence according to SEQ ID No. 1, and a second part comprising a variant of the Fc portion of human IgG1, said variant having the sequence according to SEQ ID No. 59 or a variant of the Fc portion of human IgG4, said variant having the sequence according to SEQ ID No. 60.

The second part may comprise a variant of the Fc portion of human IgG1 having the sequence according to SEQ ID No. 59 and the first part and the second part are linked by the amino acid sequence according to SEQ ID No. 15 or the second part may comprise a variant of the Fc portion of human IgG4 having the sequence according to SEQ ID No. 60 and the first part and the second part are linked by the amino acid sequence according to SEQ ID No. 4.

The fragment of human ACE2 may consist of a protein having the amino acid sequence according to SEQ ID No. 2 or may be the extracellular domain of ACE2 consisting of a protein having the amino acid sequence according to SEQ ID No. 3.

In one embodiment. the variant of the human ACE2 fragment is an enzymatically inactive variant of human ACE2.

The enzymatically inactive variant of human ACE2 may comprise a H374N and a H378N mutation, the numbering referring to SEQ ID No. 1 or a mutation at position 273, preferably a R273A mutation, the numbering referring to SEQ ID No. 1.

The present invention further provides a fusion protein comprising:
(a) a first part comprising a variant of a fragment of human ACE2, said human ACE2 having the amino acid sequence according to SEQ ID No. 1, wherein the variant of the human ACE2 fragment comprises a R273A mutation, the numbering referring to SEQ ID No. 1, and
(b) a second part comprising the Fc portion of human IgG or a variant of the Fc portion of human IgG.

In one embodiment, the variant of a fragment of human ACE2 consists of a protein having the amino acid sequence according to SEQ ID No. 24 or 25.

The second part may comprise the Fc part of human IgG1 or IgG4.

In one embodiment, the second part comprises the Fc part of human IgG1 according to SEQ ID No. 16, preferably the first part and the second part are linked by the amino acid sequence according to SEQ ID No. 15.

In an alternative embodiment, the second part comprises the Fc part of human IgG4 according to SEQ ID No. 5, preferably the first part and the second part are linked by the amino acid sequence according to SEQ ID No. 4.

In an alternative embodiment, the second part comprises a variant of the Fc portion of human IgG4 having the sequence according to SEQ ID No. 44.

The fusion protein of the present invention may have an amino acid sequence selected from the group of amino acid sequences according to SEQ ID Nos. 20, 21, 22, 23, 26, 27, 30, 31, 34, 35, 38, 39, 40, 41, 42, 43, 45, 46, 47, 48, 49, 50, 51, 52, 53, 54, 55, 56, 57, 58, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 93 and 94.

The present invention also relates to a nucleic acid molecule comprising a nucleic acid sequence encoding said fusion protein, an expression vector comprising said nucleic acid molecule and a host cell comprising said nucleic acid molecule or said expression vector.

Further, the present invention relates to a method for producing said fusion protein, comprising culturing said host cell in a suitable culture medium.

The present invention also relates to said fusion protein for medical use, in particular for use in preventing and/or treating an infection with a coronavirus binding to ACE2.

In one embodiment, the coronavirus binding to ACE2 is selected from the group consisting of SARS, SARS-CoV-2 and NL-63, preferably is SARS-CoV-2.

Said fusion protein may be administered in combination with an anti-viral agent which may be selected from the group consisting of remdesivir, arbidol HCl, ritonavir, lopinavir, darunavir, ribavirin, chloroquin and derivatives thereof, nitazoxanide, camostat mesilate, tocilizumab, siltuximab, sarilumab and baricitinib phosphate.

Said fusion protein may also be administered in combination with an antibody which may be selected from the group consisting of bamlanivimab, etesevimab, casirivimab, imdevimab, sotrovimab and mixtures thereof.

The present invention also relates to said fusion protein for use in treating hypertension (including high blood pressure), congestive heart failure, chronic heart failure, acute heart failure, contractile heart failure, myocardial infarction, arteriosclerosis, kidney failure, renal failure, Acute Respiratory Distress Syndrome (ARDS), Acute Lung Injury (ALI), chronic obstructive pulmonary disease (COPD), pulmonary hypertension, renal fibrosis, chronic renal failure, acute renal failure, acute kidney injury, inflammatory bowel disease and multi-organ dysfunction syndrome.

The present invention also relates to a pharmaceutical composition comprising said fusion protein and a pharmaceutically acceptable carrier or excipient. The pharmaceutical composition may further comprise an anti-viral agent.

### BRIEF DESCRIPTION OF THE DRAWINGS

FIG. 1: Protein yield of different fusion proteins after protein A chromatography as determined by slope spectroscopy

FIG. 2: Analysis of high molecular weight species of different fusion proteins by analytical size exclusion chromatography

FIG. 3: Analysis of O-glycosylation of different fusion proteins

FIG. 4: Inhibition of S1 binding to ACE2 by different fusion proteins as determined by competitive ELISA
a) Constructs 1-4 having the Fc part of IgG4
b) Constructs 5-8 having the Fc part of IgG1

FIG. 5: Neutralization of SARS-CoV-2 (strain Victoria/1/2020) by constructs 1, 3, 5 and 7

FIG. 6: Analysis of enzymatic activity of constructs 1 to 8 and two reference proteins (Ref1, Ref2)
a) Mean values of the enzymatic activity with standard deviation from six individual experiments
b) Single values for each construct obtained in six individual experiments

FIG. 7: Neutralization of different coronaviruses by constructs 1 to 8
a) Neutralization of SARS-CoV (strain SARS-CoV-Fra-1 (AY291315.1)); mean IC50 values from three independent experiments; error bars indicate 95% confidence interval
b) Neutralization of SARS-CoV-2 (SARS-CoV-2-Munich-TUM-1 (EPI_ISL_582134)); mean IC50 values from three independent experiments; error bars indicate 95% confidence interval
c) Neutralization of SARS-CoV-2 D614G; mean IC50 values from three independent experiments;error bars indicate 95% confidence interval

FIG. 8: Binding of the fusion protein according to SEQ ID NO. 6 to ACE2 as determined by binding ELISA

FIG. 9: Neutralization of different virus isolates (A: SARS-CoV-2 Munich-TUM-1; B: SARS-CoV-2 D614G; C: SARS-CoV-2 B.1.1.7; D: SARS-CoV-2 B.1.351) with the fusion proteins according to SEQ ID No. 6 (construct 1, left-hand side) and SEQ ID No. 8 (construct 3, right-hand side). The dotted line depicts the determination of the IC50 value. Data given are means±SEM of three independent experiments each.

FIG. 10: Analysis of high molecular weight species of different fusion proteins after storage for different periods by analytical size exclusion chromatography
a) Analysis of constructs 1 to 4 (according to SEQ ID NOs. 6 to 9)
b) Analysis of constructs 5 to 8 (according to SEQ ID NOs. 10 to 13)

### DETAILED DESCRIPTION OF THE INVENTION

The present invention as illustratively described in the following may suitably be practiced in the absence of any element or elements, limitation or limitations, not specifically disclosed herein.

The present invention will be described with respect to particular embodiments, but the invention is not limited thereto, but only by the claims.

Where the term “comprising” is used in the present description and claims, it does not exclude other elements. For the purposes of the present invention, the term “consisting of” is considered to be a preferred embodiment of the term “comprising”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. a cell or organism is defined to be obtainable by a specific method, this is also to be understood to disclose a cell or organism which is obtained by this method.

Where an indefinite or definite article is used when referring to a singular noun, e.g. “a”, “an” or “the”, this includes a plural of that noun unless something else is specifically stated.

As discussed above, the present invention provides a fusion protein comprising a first part comprising certain variants of fragment of human ACE2 and a second part comprising the Fc portion of a human IgG or IgG1 or a variant of the Fc portion of human IgG or IgG1. The present invention also provides a fusion protein comprising a first part comprising a certain fragment of human ACE2 or a variant of said fragment and a second part comprising a variant of the Fc portion of human IgG1 or human IgG4. It was shown that a fragment of human ACE2 comprising amino acids 18 to 732 provides a higher yield and a lower amount of high molecular weight species than a fragment of human ACE2 comprising amino acids 18 to 740 as well as no non-natural O-glycosylation, as serine 740 is missing. Further, the fragment of human ACE2 comprising amino acids 18 to 732 showed a higher stability after storage than a fragment of human ACE2 comprising amino acids 18 to 740. Nevertheless, a fusion protein with the fragment of human ACE2 comprising amino acids 18 to 732 neutralized different SARS-CoV2 variants with a similar efficiency as a fusion protein with the fragment of human ACE2 comprising amino acids 18 to 740. In contrast, it has been found by others that a fusion protein comprising only amino acids 18 to 615 of ACE2 blocked infection with SARS-CoV2 less efficiently than a fusion protein comprising amino acids 18 to 740 of ACE2 (Li et al. (2020) J. Virol. 94(22): e01283-20). In view of the literature (e.g. Tada et al. (2020), available at https://www.biorxiv.org/content/10.1101/2020.09.16.300319v1.full) it is assumed that a fusion protein with the Fc part of human IgG has a higher affinity to the Spike protein of SARS-CoV-2 than a soluble ACE2 dimer without the Fc portion. It is also assumed that the disulfide bridge between the Fc portions stabilizes the binding of the fusion proteins to their target, such as the Spike protein of SARS-CoV-2. The fusion protein of the present invention binds to FcRn which leads to a longer half-life period than the soluble ACE2 dimer.

It was shown that Fc-mediated antibody functions such as antibody-dependent cytotoxicity (ADCC) and antibody-dependent phagocytosis (ADP) protect against influenza virus and other viral pathogens (Vanderven and Kent (2020) Immunology & Cell Biology 98: 253-263). In the context of SARS-CoV2 treatment the EMA Assessment Report for the mixture of the antibodies casirivimab and imdevimab discusses that these antibodies are able to mediate ADCC and ADP. Further, no evidence of antibody-dependent enhancement was observed in any treated animal.

A “fusion protein” is a protein which is formed by at least two polypeptide parts which are not naturally linked with each other. The two polypeptide parts are linked by a peptide bond and optionally a linker molecule is inserted between the two polypeptide parts. The two polypeptide parts are transcribed and translated as a single molecule. The fusion protein typically has functionalities derived from both polypeptide parts. In the context of the present invention, the fusion protein retains the binding properties of ACE2, in particular the binding of viruses such as coronaviruses, and the increased half-life and Fc receptor binding conferred by the Fc portion of human IgG.

The term “human ACE2” refers to angiotensin converting enzyme 2 derived from a human subject. The full-length sequence of human ACE2 has 805 amino acids. It comprises a signal peptide, an N-terminal extracellular peptidase domain followed by a collectrin-like domain, a single transmembrane helix and a short intracellular segment. The full-length sequence of human ACE2 is depicted in SEQ ID No. 1. Unless indicated otherwise, the amino acid numbering used herein refers to the numbering of the full-length sequence of human ACE2 according to SEQ ID No. 1. The extracellular domain of human ACE2 consists of the amino acids 18 to 740 of SEQ ID No. 1 and is depicted in SEQ ID No. 3.

The term “fragment of human ACE2” refers to a polypeptide which lacks one or more amino acids compared to the full-length sequence of human ACE2 according to SEQ ID No. 1. The fragment of human ACE2 is capable of binding to the S protein of at least one coronavirus, in particular to the S protein of SARS-CoV-2. The binding of a fragment of human ACE2 to the S protein of at least one coronavirus can be determined in an ELISA assay in which the S protein is immobilized on a substrate and contacted with the fragment of human ACE2 and the interaction between the S protein and the fragment of human ACE2 is detected. Alternatively, the binding of a fragment of human ACE2 to the S protein of at least one coronavirus can be determined by surface plasmon resonance, e.g. as described in Shang et al. (2020) Nature doi: 10.1038/s41586-020-2179-y; Wrapp et al. (2020) Science 367(6483): 1260-1263; Lei et al. (2020) Nature Communications 11(1): 2070. In a further alternative, the binding of a fragment of human ACE2 to the S protein of at least one coronavirus can be determined by biolayer interferometry, e.g. as described in Seydoux et al. (2020) https://doi.org/10.1101/2020.05.12.091298. Suitable methods for determining the binding to the S protein are also described in the examples section herein.

In one embodiment, the fragment of human ACE2 consists of 360 to 723 contiguous amino acids within the sequence according to SEQ ID No. 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID No. 1. Preferably, the fragment of human ACE2 consists of 520 to 723, 540 to 723, 560 to 723, 580 to 723 or 600 to 723 contiguous amino acids within the sequence according to SEQ ID No. 1. More preferably, the fragment of human ACE2 consists of 620 to 723, 640 to 723, 660 to 723, 680 to 723, 700 to 723 or 720 to 723 contiguous amino acids within the sequence according to SEQ ID No. 1.

In one embodiment, the fragment of human ACE2 comprises the amino acid residues K31 and K353, the numbering referring to SEQ ID No. 1. In one embodiment, the fragment of human ACE2 comprises the amino acid residues Q24, D30, E35 and Q42, the numbering referring to SEQ ID No. 1. In one embodiment, the fragment of human ACE2 comprises the amino acid residues Q24, D30, K31, E35, Q42 and K353, the numbering referring to SEQ ID No. 1.

In one embodiment, the fragment of human ACE2 consists of 360 to 723 contiguous amino acids within the sequence according to SEQ ID No. 1 and comprises the amino acid residues K31 and K353, the numbering referring to SEQ ID No. 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID No. 1 and comprises the amino acid residues K31 and K353, the numbering referring to SEQ ID No. 1. Preferably, the fragment of human ACE2 consists of 520 to 723, 540 to 723, 560 to 723, 580 to 723 or 600 to 723 contiguous amino acids within the sequence according to SEQ ID No. 1 and comprises the amino acid residues K31 and K353, the numbering referring to SEQ ID No. 1. More preferably, the fragment of human ACE2 consists of 620 to 723, 640 to 723, 660 to 723, 680 to 723, 700 to 723 or 720 to 723 contiguous amino acids within the sequence according to SEQ ID No. 1 and comprises the amino acid residues K31 and K353, the numbering referring to SEQ ID No. 1.

In one embodiment, the fragment of human ACE2 consists of 360 to 723 contiguous amino acids within the sequence according to SEQ ID No. 1 and comprises the amino acid residues Q24, D30, E35 and Q42, the numbering referring to SEQ ID No. 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID No. 1 and comprises the amino acid residues Q24, D30, E35 and Q42, the numbering referring to SEQ ID No. 1. Preferably, the fragment of human ACE2 consists of 520 to 723, 540 to 723, 560 to 723, 580 to 723 or 600 to 723 contiguous amino acids within the sequence according to SEQ ID No. 1 and comprises the amino acid residues Q24, D30, E35 and Q42, the numbering referring to SEQ ID No. 1. More preferably, the fragment of human ACE2 consists of 620 to 723, 640 to 723, 660 to 723, 680 to 723, 700 to 723 or 720 to 723 contiguous amino acids within the sequence according to SEQ ID No. 1 and comprises the amino acid residues Q24, D30, E35 and Q42, the numbering referring to SEQ ID No. 1.

In one embodiment, the fragment of human ACE2 consists of 360 to 723 contiguous amino acids within the sequence according to SEQ ID No. 1 and comprises the amino acid residues Q24, D30, K31, E35, Q42 and K353, the numbering referring to SEQ ID No. 1. Preferably, the fragment of human ACE2 consists of 380 to 723, 400 to 723, 420 to 723, 440 to 723, 460 to 723, 480 to 723 or 500 to 723 contiguous amino acids within the sequence according to SEQ ID No. 1 and comprises the amino acid residues Q24, D30, K31, E35, Q42 and K353, the numbering referring to SEQ ID No. 1. Preferably, the fragment of human ACE2 consists of 520 to 723, 540 to 723, 560 to 723, 580 to 723 or 600 to 723 contiguous amino acids within the sequence according to SEQ ID No. 1 and comprises the amino acid residues Q24, D30, K31, E35, Q42 and K353, the numbering referring to SEQ ID No. 1. More preferably, the fragment of human ACE2 consists of 620 to 723, 640 to 723, 660 to 723, 680 to 723, 700 to 723 or 720 to 723 contiguous amino acids within the sequence according to SEQ ID No. 1 and comprises the amino acid residues Q24, D30, K31, E35, Q42 and K353, the numbering referring to SEQ ID No. 1.

In one embodiment, the fragment of human ACE2 consists of amino acids 18 to 380, 18 to 400, 18 to 420, 18 to 440, 18 to 460, 18 to 480 or 18 to 500 of the sequence according to SEQ ID No. 1. Preferably, the fragment of human ACE2 consists of amino acids 18 to 520, 18 to 540, 18 to 560, 18 to 580 or 18 to 600 of the sequence according to SEQ ID No. 1. More preferably, the fragment of human ACE2 consists of amino acids 18 to 605, 18 to 615, 18 to 620, 18 to 640, 18 to 660, 18 to 680 or 18 to 700 of the sequence according to SEQ ID No. 1. Even more preferably, the fragment of human ACE2 consists of amino acids 18 to 710, 18 to 720 or 18 to 730 of the sequence according to SEQ ID No. 1.

In one embodiment, the fragment of human ACE2 consists of the amino acid sequence according to SEQ ID No. 2. The amino acid sequence according to SEQ ID No. 2 starts at amino acid Q18 and ends with amino acid G732 in the sequence according to SEQ ID No. 1. The amino acid glycine at the C-terminal end of this fragment provides a high rotational freedom which favours the fusion of the two protein parts and increases the stability of the fusion protein. Additionally, it has surprisingly been found that the use of an ACE2 fragment comprising the amino acid sequence which starts at amino acid Q18 and ends with amino acid G732 in the sequence according to SEQ ID No. 1 provides a better yield as well as a better stability than a longer ACE2 fragment. Moreover, it lacks a non-natural O glycosylation and neutralizes different SARS-CoV2 variants with similar efficiency as the longer ACE2 fragment. This is in contrast to findings with a shorter ACE2 fragment comprising only amino acids 18 to 615 of ACE2 which inhibited infection with SARS-CoV2 less efficiently as the longer ACE2 fragment comprising amino acids 18 to 740 of ACE2.

In one embodiment, the fragment of human ACE2 consists of the complete extracellular domain of human ACE2 which has the amino acid sequence according to SEQ ID No. 3.

In one embodiment, the fragment of human ACE2 consists of the amino acid sequence according to SEQ ID No. 14. The amino acid sequence according to SEQ ID No. 14 starts at amino acid Q18 and ends with amino acid G605 in the sequence according to SEQ ID No. 1.

In one embodiment, the fragment of human ACE2 is N-glycosylated at at least one amino acid residue selected from N53, N90, N103, N322, N432, N546 and N690, the numbering referring to SEQ ID No. 1. In one embodiment, the fragment of human ACE2 is N-glycosylated at amino acid residues N53, N90 and, N322, the numbering referring to SEQ ID No. 1. In one embodiment, the fragment of human ACE2 is N-glycosylated at amino acid residues N53, N90, N103, N322, N432, N546 and N690, the numbering referring to SEQ ID No. 1.

In one embodiment, the fragment of human ACE2 consists of the amino acid sequence according to SEQ ID No. 2 and is N-glycosylated at at least one amino acid residue selected from N53, N90, N103, N322, N432, N546 and N690, the numbering referring to SEQ ID No. 1. In one embodiment, the fragment of human ACE2 consists of the amino acid sequence according to SEQ ID No. 2 and is N-glycosylated at amino acid residues N53, N90 and N322, the numbering referring to SEQ ID No. 1. In one embodiment, the fragment of human ACE2 consists of the amino acid sequence according to SEQ ID No. 2 and is N-glycosylated at amino acid residues N53, N90, N103, N322, N432, N546 and N690, the numbering referring to SEQ ID No. 1.

In one embodiment, the fragment of human ACE2 consists of the amino acid sequence according to SEQ ID No. 3 and is N-glycosylated at at least one amino acid residue selected from N53, N90, N103, N322, N432, N546 and N690, the numbering referring to SEQ ID No. 1. In one embodiment, the fragment of human ACE2 consists of the amino acid sequence according to SEQ ID No. 3 and is N-glycosylated at amino acid residues N53, N90 and N322, the numbering referring to SEQ ID No. 1. In one embodiment, the fragment of human ACE2 consists of the amino acid sequence according to SEQ ID No. 3 and is N-glycosylated at amino acid residues N53, N90, N103, N322, N432, N546 and N690, the numbering referring to SEQ ID No. 1.

In the context of the present invention, the term “fragment of human ACE2 consists of/consisting of a protein having the amino acid sequence according to SEQ ID No. 2 is intended to mean that the fragment has exactly the length as defined by the corresponding sequence in the sequence listing and does not comprise any amino acid additions and/or deletions. However, this term does not exclude the presence of amino acid substitution(s) within the sequence of the defined fragment.

In one embodiment, the fragment of human ACE2 comprises or is identified by 360 to 723 contiguous amino acids within the sequence according to SEQ ID No. 1. Preferably, the fragment of human ACE2 comprises or is identified by 380 to 723, 400 to 723, 420 to 723, 440 to 723, 460 to 723, 480 to 723 or 500 to 723 contiguous amino acids within the sequence according to SEQ ID No. 1. Preferably, the fragment of human ACE2 comprises or is identified by 520 to 723, 540 to 723, 560 to 723, 580 to 723 or 600 to 723 contiguous amino acids within the sequence according to SEQ ID No. 1. More preferably, the fragment of human ACE2 comprises or is identified by 620 to 723, 640 to 723, 660 to 723, 680 to 723, 700 to 723 or 720 to 723 contiguous amino acids within the sequence according to SEQ ID No. 1.

In one embodiment, the fragment of human ACE2 comprises the amino acid residues K31 and K353, the numbering referring to SEQ ID No. 1. In one embodiment, the fragment of human ACE2 comprises the amino acid residues Q24, D30, E35 and Q42, the numbering referring to SEQ ID No. 1. In one embodiment, the fragment of human ACE2 comprises the amino acid residues Q24, D30, K31, E35, Q42 and K353, the numbering referring to SEQ ID No. 1.

In one embodiment, the fragment of human ACE2 comprises or is identified by 360 to 723 contiguous amino acids within the sequence according to SEQ ID No. 1 and comprises the amino acid residues K31 and K353, the numbering referring to SEQ ID No. 1. Preferably, the fragment of human ACE2 comprises or is identified by 380 to 723, 400 to 723, 420 to 723, 440 to 723, 460 to 723, 480 to 723 or 500 to 723 contiguous amino acids within the sequence according to SEQ ID No. 1 and comprises the amino acid residues K31 and K353, the numbering referring to SEQ ID No. 1. Preferably, the fragment of human ACE2 comprises or is identified by 520 to 723, 540 to 723, 560 to 723, 580 to 723 or 600 to 723 contiguous amino acids within the sequence according to SEQ ID No. 1 and comprises the amino acid residues K31 and K353, the numbering referring to SEQ ID No. 1. More preferably, the fragment of human ACE2 comprises or is identified by 620 to 723, 640 to 723, 660 to 723, 680 to 723, 700 to 723 or 720 to 723 contiguous amino acids within the sequence according to SEQ ID No. 1 and comprises the amino acid residues K31 and K353, the numbering referring to SEQ ID No. 1.

In one embodiment, the fragment of human ACE2 comprises or is identified by 360 to 723 contiguous amino acids within the sequence according to SEQ ID No. 1 and comprises the amino acid residues Q24, D30, E35 and Q42, the numbering referring to SEQ ID No. 1. Preferably, the fragment of human ACE2 comprises or is identified by 380 to 723, 400 to 723, 420 to 723, 440 to 723, 460 to 723, 480 to 723 or 500 to 723 contiguous amino acids within the sequence according to SEQ ID No. 1 and comprises the amino acid residues Q24, D30, E35 and Q42, the numbering referring to SEQ ID No. 1. Preferably, the fragment of human ACE2 comprises or is identified by 520 to 723, 540 to 723, 560 to 723, 580 to 723 or 600 to 723 contiguous amino acids within the sequence according to SEQ ID No. 1 and comprises the amino acid residues Q24, D30, E35 and Q42, the numbering referring to SEQ ID No. 1. More preferably, the fragment of human ACE2 comprises or is identified by 620 to 723, 640 to 723, 660 to 723, 680 to 723, 700 to 723 or 720 to 723 contiguous amino acids within the sequence according to SEQ ID No. 1 and comprises the amino acid residues Q24, D30, E35 and Q42, the numbering referring to SEQ ID No. 1.

In one embodiment, the fragment of human ACE2 comprises or is identified by 360 to 723 contiguous amino acids within the sequence according to SEQ ID No. 1 and comprises the amino acid residues Q24, D30, K31, E35, Q42 and K353, the numbering referring to SEQ ID No. 1. Preferably, the fragment of human ACE2 comprises or is identified by 380 to 723, 400 to 723, 420 to 723, 440 to 723, 460 to 723, 480 to 723 or 500 to 723 contiguous amino acids within the sequence according to SEQ ID No. 1 and comprises the amino acid residues Q24, D30, K31, E35, Q42 and K353, the numbering referring to SEQ ID No. 1. Preferably, the fragment of human ACE2 comprises or is identified by 520 to 723, 540 to 723, 560 to 723, 580 to 723 or 600 to 723 contiguous amino acids within the sequence according to SEQ ID No. 1 and comprises the amino acid residues Q24, D30, K31, E35, Q42 and K353, the numbering referring to SEQ ID No. 1. More preferably, the fragment of human ACE2 comprises or is identified by 620 to 723, 640 to 723, 660 to 723, 680 to 723, 700 to 723 or 720 to 723 contiguous amino acids within the sequence according to SEQ ID No. 1 and comprises the amino acid residues Q24, D30, K31, E35, Q42 and K353, the numbering referring to SEQ ID No. 1.

In one embodiment, the fragment of human ACE2 comprises or is identified by amino acids 18 to 380, 18 to 400, 18 to 420, 18 to 440, 18 to 460, 18 to 480 or 18 to 500 of the sequence according to SEQ ID No. 1. Preferably, the fragment of human ACE2 comprises or is identified by amino acids 18 to 520, 18 to 540, 18 to 560, 18 to 580 or 18 to 600 of the sequence according to SEQ ID No. 1. More preferably, the fragment of human ACE2 comprises or is identified by amino acids 18 to 605, 18 to 615, 18 to 620, 18 to 640, 18 to 660, 18 to 680 or 18 to 700 of the sequence according to SEQ ID No. 1. Even more preferably, the fragment of human ACE2 comprises or is identified by amino acids 18 to 710, 18 to 720 or 18 to 730 of the sequence according to SEQ ID No. 1.

In one embodiment, the fragment of human ACE2 comprises or is identified by the amino acid sequence according to SEQ ID No. 2. The amino acid sequence according to SEQ ID No. 2 starts at amino acid Q18 and ends with amino acid G732 in the sequence according to SEQ ID No. 1. The amino acid glycine at the C-terminal end of this fragment provides a high rotational freedom which favours the fusion of the two protein parts and increases the stability of the fusion protein. Additionally, the use of an ACE2 fragment comprising or which is identified by the amino acid sequence which starts at amino acid Q18 and ends with amino acid G732 in the sequence according to SEQ ID No. 1 provides a better yield than a longer ACE2 fragment.

In one embodiment, the fragment of human ACE2 comprises or is identified by the complete extracellular domain of human ACE2 which has the amino acid sequence according to SEQ ID No. 3.

In one embodiment, the fragment of human ACE2 comprises or is identified by the amino acid sequence according to SEQ ID No. 14. The amino acid sequence according to SEQ ID No. 14 starts at amino acid Q18 and ends with amino acid G605 in the sequence according to SEQ ID No. 1.

In one embodiment, the fragment of human ACE2 is N-glycosylated at at least one amino acid residue selected from N53, N90, N103, N322, N432, N546 and N690, the numbering referring to SEQ ID No. 1. In one embodiment, the fragment of human ACE2 is N-glycosylated at amino acid residues N53, N90 and, N322, the numbering referring to SEQ ID No. 1. In one embodiment, the fragment of human ACE2 is N-glycosylated at amino acid residues N53, N90, N103, N322, N432, N546 and N690, the numbering referring to SEQ ID No. 1.

In one embodiment, the fragment of human ACE2 comprises or is identified by the amino acid sequence according to SEQ ID No. 2 and is N-glycosylated at at least one amino acid residue selected from N53, N90, N103, N322, N432, N546 and N690, the numbering referring to SEQ ID No. 1. In one embodiment, the fragment of human ACE2 comprises or is identified by of the amino acid sequence according to SEQ ID No. 2 and is N-glycosylated at amino acid residues N53, N90 and N322, the numbering referring to SEQ ID No. 1. In one embodiment, the fragment of human ACE2 comprises or is identified by the amino acid sequence according to SEQ ID No. 2 and is N-glycosylated at amino acid residues N53, N90, N103, N322, N432, N546 and N690, the numbering referring to SEQ ID No. 1.

In one embodiment, the fragment of human ACE2 comprises or is identified by the amino acid sequence according to SEQ ID No. 3 and is N-glycosylated at at least one amino acid residue selected from N53, N90, N103, N322, N432, N546 and N690, the numbering referring to SEQ ID No. 1. In one embodiment, the fragment of human ACE2 comprises or is identified by the amino acid sequence according to SEQ ID No. 3 and is N-glycosylated at amino acid residues N53, N90 and N322, the numbering referring to SEQ ID No. 1. In one embodiment, the fragment of human ACE2 comprises or is identified by the amino acid sequence according to SEQ ID No. 3 and is N-glycosylated at amino acid residues N53, N90, N103, N322, N432, N546 and N690, the numbering referring to SEQ ID No. 1.

The term “N-glycosylated” or “N-glycosylation” means that a glycan structure is attached to the amide nitrogen of an asparagine residue of a protein. A glycan is a branched, flexible chain of carbohydrates and the exact structure of the glycan attached to the asparagine residue of a protein depends on the expression system used for glycoprotein production.

A “variant” of the fragment of human ACE2 refers to a fragment as defined above, wherein compared to the corresponding sequence in the amino acid sequence of wild-type, full-length human ACE2 according to SEQ ID No. 1 at least one amino acid residue is different or at least two, at least three, at least four, at least five, at least six, at least seven, at least eight, at least nine, at least ten, at least eleven or at least thirteen amino acids are different. The “variant” of the fragment of human ACE2 comprises one or more amino acid substitutions in the sequence of the fragment of human ACE2. A “variant” of the fragment of human ACE2 does not comprise any amino acid additions or deletions compared to the sequence from which the variant is derived. In one embodiment, the variant the fragment of human ACE2 is a variant of the fragment of human ACE2 according to SEQ ID No. 2 or 3 and does not comprise any amino acid additions or deletions compared to the sequence according to SEQ ID No. 2 or 3, i.e. it has the same length as the sequence according to SEQ ID No. 2 or 3. Within the scope of the present invention a variant of the fragment of human ACE2 is capable of binding to the S protein of at least one coronavirus, in particular to the S protein of SARS-CoV-2. The binding of a variant of the fragment of human ACE2 to the S protein of at least one coronavirus, in particular to the S protein of SARS-CoV-2, can be determined as described above for fragments of human ACE2.

In one embodiment, the variant of the fragment of human ACE2 differs from the corresponding amino acid sequence in the sequence according to SEQ ID No. 1 by one amino acid. In another embodiment, the variant of the fragment of human ACE2 differs from the corresponding amino acid sequence in the sequence according to SEQ ID No. 1 by two amino acids. In still another embodiment, the variant of the fragment of human ACE2 differs from the corresponding amino acid sequence in the sequence according to SEQ ID No. 1 by three amino acids. In still another embodiment, the variant of the fragment of human ACE2 differs from the corresponding amino acid sequence in the sequence according to SEQ ID No. 1 by four amino acids. In still another embodiment, the variant of the fragment of human ACE2 differs from the corresponding amino acid sequence in the sequence according to SEQ ID No. 1 by five amino acids. In still another embodiment, the variant of the fragment of human ACE2 differs from the corresponding amino acid sequence in the sequence according to SEQ ID No. 1 by six amino acids. In still another embodiment, the variant of the fragment of human ACE2 differs from the corresponding amino acid sequence in the sequence according to SEQ ID No. 1 by seven amino acids. In still another embodiment, the variant of the fragment of human ACE2 differs from the corresponding amino acid sequence in the sequence according to SEQ ID No. 1 by eight amino acids. In still another embodiment, the variant of the fragment of human ACE2 differs from the corresponding amino acid sequence in the sequence according to SEQ ID No. 1 by nine amino acids. In still another embodiment, the variant of the fragment of human ACE2 differs from the corresponding amino acid sequence in the sequence according to SEQ ID No. 1 by ten amino acids. In still another embodiment, the variant of the fragment of human ACE2 differs from the corresponding amino acid sequence in the sequence according to SEQ ID No. 1 by eleven amino acids. In still another embodiment, the variant of the fragment of human ACE2 differs from the corresponding amino acid sequence in the sequence according to SEQ ID No. 1 by twelve amino acids. In still another embodiment, the variant of the fragment of human ACE2 differs from the corresponding amino acid sequence in the sequence according to SEQ ID No. 1 by thirteen amino acids.

One variant of the fragment of human ACE2 may be an enzymatically inactive variant. “The enzymatically inactive variant of the fragment of human ACE2” lacks the ability to cleave angiotensin II to Ang1-7. The enzymatic activity of human ACE2 can be determined by methods known to the skilled person. Suitable kits for determining the enzymatic activity of human ACE2 are commercially available, for example from the companies BioVision or Anaspec. By using an enzymatically inactive ACE2 variant any side effects associated with the enzymatic activity of ACE2 such as effects on the cardiovascular system or the regulation of blood pressure are eliminated. Further, the risk of counterbalancing the RAS-MAS equilibrium is reduced.

The enzymatically inactive variant of the fragment of human ACE2 may comprise one or more mutations of amino acids within the catalytic centre of ACE2. In particular, the enzymatically inactive variant of the fragment of human ACE2 comprises a mutation of the wildtype histidine at residue 374 of the sequence according to SEQ ID No. 1 and/or a mutation of the wildtype histidine at residue 378 of the sequence according to SEQ ID No. 1. The wild-type histidine may be mutated to any amino acid other than histidine and particularly, the wild-type histidine is mutated to asparagine. Preferably, the enzymatically inactive variant of the fragment of human ACE2 comprises a H374N and a H378N mutation, the numbering referring to the sequence according to SEQ ID No. 1.

In another embodiment, the enzymatically inactive variant of the fragment of human ACE2 comprises a mutation at one or more of the following amino acid residues, the numbering referring to the sequence according to SEQ ID No. 1: residue 345 (histidine in wild-type), 273 (arginine in wild-type), 402 (glutamic acid in wild-type) and 505 (histidine in wild-type). In one embodiment, the enzymatically inactive variant of the fragment of human ACE2 comprises a mutation of histidine at residue 345 to alanine or leucine, a mutation of arginine at residue 273 to alanine, glutamine or lysine, a mutation of glutamic acid at residue 402 to alanine and/or a mutation of histidine at residue 505 to alanine or leucine, the numbering referring to the sequence according to SEQ ID No. 1. In a particular embodiment, the enzymatically inactive variant of the fragment of human ACE2 comprises a mutation of arginine at residue 273 to alanine (also called R273A mutation). It was found that arginine 273 is critical for substrate binding and that its substitution abolishes enzymatic activity (Guy et al. (2005) FEBS J. 272(14): 3512-3520).

In one embodiment, the fragment of human ACE2 consists of amino acids 18 to 380, 18 to 400, 18 to 420, 18 to 440, 18 to 460, 18 to 480 or 18 to 500 of the sequence according to SEQ ID No. 1 and comprises a H374N and a H378N mutation, the numbering referring to the sequence according to SEQ ID No. 1. Preferably, the fragment of human ACE2 consists of amino acids 18 to 520, 18 to 540, 18 to 560, 18 to 580 or 18 to 600 of the sequence according to SEQ ID No. 1 and comprises a H374N and a H378N mutation, the numbering referring to the sequence according to SEQ ID No. 1. More preferably, the fragment of human ACE2 consists of amino acids 18 to 615, 18 to 620, 18 to 640, 18 to 660, 18 to 680 or 18 to 700 of the sequence according to SEQ ID No. 1 and comprises a H374N and a H378N mutation, the numbering referring to the sequence according to SEQ ID No. 1. Even more preferably, the fragment of human ACE2 consists of amino acids 18 to 710, 18 to 720 or 18 to 730 of the sequence according to SEQ ID No. 1 and comprises a H374N and a H378N mutation, the numbering referring to the sequence according to SEQ ID No. 1.

In one embodiment, the fragment of human ACE2 consists of the sequence according to SEQ ID No. 2 and comprises a H374N and a H378N mutation. In one embodiment, the variant of the fragment of human ACE2 consists of a protein having the amino acid sequence according to SEQ ID No. 91.

In one embodiment, the fragment of human ACE2 consists of the sequence according to SEQ ID No. 3 and comprises a H374N and a H378N mutation. In one embodiment, the variant of the fragment of human ACE2 consists of a protein having the amino acid sequence according to SEQ ID No. 92.

In one embodiment, the fragment of human ACE2 comprises or is identified by amino acids 18 to 380, 18 to 400, 18 to 420, 18 to 440, 18 to 460, 18 to 480 or 18 to 500 of the sequence according to SEQ ID No. 1 and comprises a H374N and a H378N mutation, the numbering referring to the sequence according to SEQ ID No. 1. Preferably, the fragment of human ACE2 comprises or is identified by amino acids 18 to 520, 18 to 540, 18 to 560, 18 to 580 or 18 to 600 of the sequence according to SEQ ID No. 1 and comprises a H374N and a H378N mutation, the numbering referring to the sequence according to SEQ ID No. 1. More preferably, the fragment of human ACE2 comprises or is identified by amino acids 18 to 615, 18 to 620, 18 to 640, 18 to 660, 18 to 680 or 18 to 700 of the sequence according to SEQ ID No. 1 and comprises a H374N and a H378N mutation, the numbering referring to the sequence according to SEQ ID No. 1. Even more preferably, the fragment of human ACE2 comprises or is identified by amino acids 18 to 710, 18 to 720 or 18 to 730 of the sequence according to SEQ ID No. 1 and comprises a H374N and a H378N mutation, the numbering referring to the sequence according to SEQ ID No. 1.

In one embodiment, the fragment of human ACE2 comprises or is identified by the sequence according to SEQ ID No. 2 and comprises a H374N and a H378N mutation. In one embodiment, the fragment of human ACE2 comprises or is identified by the sequence according to SEQ ID No. 3 and comprises a H374N and a H378N mutation.

In one embodiment, the fragment of human ACE2 consists of amino acids 18 to 380, 18 to 400, 18 to 420, 18 to 440, 18 to 460, 18 to 480 or 18 to 500 of the sequence according to SEQ ID No. 1 and comprises a R273A mutation, the numbering referring to the sequence according to SEQ ID No. 1. Preferably, the fragment of human ACE2 consists of amino acids 18 to 520, 18 to 540, 18 to 560, 18 to 580 or 18 to 600 of the sequence according to SEQ ID No. 1 and comprises a R273A mutation, the numbering referring to the sequence according to SEQ ID No. 1. More preferably, the fragment of human ACE2 consists of amino acids 18 to 615, 18 to 620, 18 to 640, 18 to 660, 18 to 680 or 18 to 700 of the sequence according to SEQ ID No. 1 and comprises a R273A mutation, the numbering referring to the sequence according to SEQ ID No. 1. Even more preferably, the fragment of human ACE2 consists of amino acids 18 to 710, 18 to 720 or 18 to 730 of the sequence according to SEQ ID No. 1 and comprises a R273A mutation, the numbering referring to the sequence according to SEQ ID No. 1.

In one embodiment, the fragment of human ACE2 consists of a protein having the amino acid sequence according to SEQ ID No. 2 and comprises a R273A mutation. In one embodiment, the fragment of human ACE2 consists of a protein having the amino acid sequence according to SEQ ID No. 3 and comprises a R273A mutation. In one embodiment, the variant of the fragment of human ACE2 consists of a protein having the amino acid sequence according to SEQ ID No. 24. In one embodiment, the variant of the fragment of human ACE2 consists of a protein having the amino acid sequence according to SEQ ID No. 25.

In one embodiment, the fragment of human ACE2 comprises or is identified by amino acids 18 to 380, 18 to 400, 18 to 420, 18 to 440, 18 to 460, 18 to 480 or 18 to 500 of the sequence according to SEQ ID No. 1 and comprises a R273A mutation, the numbering referring to the sequence according to SEQ ID No. 1. Preferably, the fragment of human ACE2 comprises or is identified by amino acids 18 to 520, 18 to 540, 18 to 560, 18 to 580 or 18 to 600 of the sequence according to SEQ ID No. 1 and comprises a R273A mutation, the numbering referring to the sequence according to SEQ ID No. 1. More preferably, the fragment of human ACE2 comprises or is identified by amino acids 18 to 615, 18 to 620, 18 to 640, 18 to 660, 18 to 680 or 18 to 700 of the sequence according to SEQ ID No. 1 and comprises a R273A mutation, the numbering referring to the sequence according to SEQ ID No. 1. Even more preferably, the fragment of human ACE2 comprises or is identified by amino acids 18 to 710, 18 to 720 or 18 to 730 of the sequence according to SEQ ID No. 1 and comprises a R273A mutation, the numbering referring to the sequence according to SEQ ID No. 1.

In one embodiment, the fragment of human ACE2 comprises or is identified by the sequence according to SEQ ID No. 2 and comprises a R273A mutation. In one embodiment, the fragment of human ACE2 comprises or is identified by the sequence according to SEQ ID No. 3 and comprises a R273A mutation. In one embodiment, the variant of the fragment of human ACE2 comprises or is identified by the sequence according to SEQ ID No. 24 or 25.

Another variant of the fragment of human ACE2 may be a variant which inhibits shedding of ACE2. It was shown that ACE2 is shed from human airway epithelia by cleavage of the ACE2 ectodomain and that ADAM17 regulates ACE2 cleavage. Further, a point mutation at leucine 584 of full-length ACE2 which is located in the ectodomain of ACE2 abolished shedding (Jia et al. (2009) Am. J. Physiol. Lung Cell. Mol. Physiol. 297(1): L84-96). Hence, in one embodiment the variant of the fragment of human ACE2 comprises a mutation at leucine 584, the numbering referring to the sequence according to SEQ ID No. 1. In one embodiment the mutation at leucine 584 is a L584A mutation.

In one embodiment, the fragment of human ACE2 consists of the sequence according to SEQ ID No. 2 and comprises a L584A mutation, the numbering referring to the sequence according to SEQ ID No. 1.

In one embodiment, the fragment of human ACE2 consists of the sequence according to SEQ ID No. 3 and comprises a L584A mutation, the numbering referring to the sequence according to SEQ ID No. 1.

In one embodiment, the fragment of human ACE2 comprises or is identified by the sequence according to SEQ ID No. 2 and comprises a L584A mutation, the numbering referring to the sequence according to SEQ ID No. 1.

In one embodiment, the fragment of human ACE2 comprises or is identified by the sequence according to SEQ ID No. 3 and comprises a L584A mutation, the numbering referring to the sequence according to SEQ ID No. 1.

In one embodiment, the variant of the fragment of human ACE2 comprises a H374N mutation, a H378N mutation and a L584A mutation, the numbering referring to the sequence according to SEQ ID No. 1.

In one embodiment, the fragment of human ACE2 consists of the sequence according to SEQ ID No. 2 and comprises a H374N mutation, a H378N mutation and a L584A mutation, the numbering referring to the sequence according to SEQ ID No. 1.

In one embodiment, the fragment of human ACE2 consists of the sequence according to SEQ ID No. 3 and comprises a H374N mutation, a H378N mutation and a L584A mutation, the numbering referring to the sequence according to SEQ ID No. 1.

In one embodiment, the fragment of human ACE2 comprises or is identified by the sequence according to SEQ ID No. 2 and comprises a H374N mutation, a H378N mutation and a L584A mutation, the numbering referring to the sequence according to SEQ ID No. 1.

In one embodiment, the fragment of human ACE2 comprises or is identified by the sequence according to SEQ ID No. 3 and comprises a H374N mutation, a H378N mutation and a L584A mutation, the numbering referring to the sequence according to SEQ ID No. 1.

In one embodiment, the variant of the fragment of human ACE2 comprises a R273A mutation and a L584A mutation, the numbering referring to the sequence according to SEQ ID No. 1.

In one embodiment, the fragment of human ACE2 consists of the protein having the amino acid sequence according to SEQ ID No. 2 and comprises a R273A mutation and a L584A mutation, the numbering referring to the sequence according to SEQ ID No. 1.

In one embodiment, the fragment of human ACE2 consists of the protein having the amino acid sequence according to SEQ ID No. 3 and comprises a R273A mutation and a L584A mutation, the numbering referring to the sequence according to SEQ ID No. 1.

In one embodiment, the fragment of human ACE2 comprises or is identified by the sequence according to SEQ ID No. 2 and comprises a R273A mutation and a L584A mutation, the numbering referring to the sequence according to SEQ ID No. 1.

In one embodiment, the fragment of human ACE2 comprises or is identified by the sequence according to SEQ ID No. 3 and comprises a R273A mutation and a L584A mutation, the numbering referring to the sequence according to SEQ ID No. 1.

Another variant of the fragment of human ACE2 may be a variant which inhibits cleavage of ACE2 by the protease TMPRSS2. It was shown that ACE2 proteolysis by TMPRSS2 augments entry of SARS-CoV (Heurich et al. (2014) J. Virol. 88(2): 1293-1307). TMPRSS2 also plays a role in the entry of SARS-CoV-2 into the cells (Hoffmann et al. (2020) Cell 181: 1-10). To abolish cleavage of ACE2 by TMPRSS2, the amino acid residues essential for the cleavage may be mutated. It was shown that arginine and lysine residues within the amino acid region spanning amino acids 697 to 716 of ACE2 are essential for ACE2 cleavage by TMPRSS2 (Heurich et al. (2014) J. Virol. 88(2): 1293-1307). Hence, in one embodiment the variant of the fragment of human ACE2 comprises a mutation at at least one residue selected from amino acids 697, 702, 705, 708, 710 and 716, the numbering referring to SEQ ID No. 1. Preferably, the variant of the fragment of human ACE2 comprises a mutation at at least two or three residues selected from amino acids 697, 702, 705, 708, 710 and 716, the numbering referring to SEQ ID No. 1. More preferably, the variant of the fragment of human ACE2 comprises a mutation at at least four or five residues selected from amino acids 697, 702, 705, 708, 710 and 716, the numbering referring to SEQ ID No. 1. Most preferably, the variant of the fragment of human ACE2 comprises a mutation at residues 697, 702, 705, 708, 710 and 716, the numbering referring to SEQ ID No. 1. The wild-type amino acid residue at any of these residues may be mutated to any other amino acid and particularly, the wild-type amino acid residue is mutated to alanine.

In one embodiment, the variant of the fragment of human ACE2 comprises at least one of the following mutations: R697A, K702A, R705A, R708A, R710A and R716A, the numbering referring to SEQ ID No. 1. Preferably, the variant of the fragment of human ACE2 comprises at least two or three of the following mutations: R697A, K702A, R705A, R708A, R710A and R716A, the numbering referring to SEQ ID No. 1. More preferably, the variant of the fragment of human ACE2 comprises at least four or five of the following mutations: R697A, K702A, R705A, R708A, R710A and R716A, the numbering referring to SEQ ID No. 1. Most preferably, the variant of the fragment of human ACE2 comprises the following mutations: R697A, K702A, R705A, R708A, R710A and R716A, the numbering referring to SEQ ID No. 1.

The variant of the fragment of human ACE2 may further comprise mutations at residues 619, 621 and/or 625, the numbering referring to SEQ ID No. 1. In particular, the variant of the fragment of human ACE2 may further comprise the following mutations: K619A, R621A and/or K625A, the numbering referring to SEQ ID No. 1.

Hence, in one embodiment, the variant of the fragment of human ACE2 comprises the following mutations: K619A, R621A, K625A, R697A, K702A, R705A, R708A, R710A and R716A, the numbering referring to SEQ ID No. 1.

In one embodiment, the fragment of human ACE2 consists of the sequence according to SEQ ID No. 2 and comprises a mutation at at least one residue selected from amino acids 697, 702, 705, 708, 710 and 716, the numbering referring to SEQ ID No. 1. In one embodiment, the fragment of human ACE2 consists of the sequence according to SEQ ID No. 2 and comprises a mutation at residues 697, 702, 705, 708, 710 and 716, the numbering referring to SEQ ID No. 1. In one embodiment, the fragment of human ACE2 consists of the sequence according to SEQ ID No. 2 and comprises at least one of the following mutations: R697A, K702A, R705A, R708A, R710A and R716A, the numbering referring to SEQ ID No. 1. In one embodiment, the fragment of human ACE2 consists of the sequence according to SEQ ID No. 2 and comprises the following mutations: R697A, K702A, R705A, R708A, R710A and R716A, the numbering referring to SEQ ID No. 1.

In one embodiment, the fragment of human ACE2 consists of the sequence according to SEQ ID No. 3 and comprises a mutation at at least one residue selected from amino acids 697, 702, 705, 708, 710 and 716, the numbering referring to SEQ ID No. 1. In one embodiment, the fragment of human ACE2 consists of the sequence according to SEQ ID No. 3 and comprises a mutation at residues 697, 702, 705, 708, 710 and 716, the numbering referring to SEQ ID No. 1. In one embodiment, the fragment of human ACE2 consists of the sequence according to SEQ ID No. 3 and comprises at least one of the following mutations: R697A, K702A, R705A, R708A, R710A and R716A, the numbering referring to SEQ ID No. 1. In one embodiment, the fragment of human ACE2 consists of the sequence according to SEQ ID No. 3 and comprises the following mutations: R697A, K702A, R705A, R708A, R710A and R716A, the numbering referring to SEQ ID No. 1.

In one embodiment, the fragment of human ACE2 consists of the sequence according to SEQ ID No. 2 and comprises a H374N mutation, a H378N mutation, a L584A mutation and the following mutations: R697A, K702A, R705A, R708A, R710A and R716A, the numbering referring to the sequence according to SEQ ID No. 1.

In one embodiment, the fragment of human ACE2 consists of the sequence according to SEQ ID No. 3 and comprises a H374N mutation, a H378N mutation, a L584A mutation and the following mutations: R697A, K702A, R705A, R708A, R710A and R716A, the numbering referring to the sequence according to SEQ ID No. 1.

In one embodiment, the fragment of human ACE2 consists of the protein having the amino acid sequence according to SEQ ID No. 2 and comprises a R273A mutation, a L584A mutation and the following mutations: R697A, K702A, R705A, R708A, R710A and R716A, the numbering referring to the sequence according to SEQ ID No. 1.

In one embodiment, the fragment of human ACE2 consists of the protein having the amino acid sequence according to SEQ ID No. 3 and comprises a R273A mutation, a L584A mutation and the following mutations: R697A, K702A, R705A, R708A, R710A and R716A, the numbering referring to the sequence according to SEQ ID No. 1.

In one embodiment, the fragment of human ACE2 consists of the sequence according to SEQ ID No. 2 and comprises a mutation at at least one residue selected from amino acids 619, 621, 625, 697, 702, 705, 708, 710 and 716, the numbering referring to SEQ ID No. 1. In one embodiment, the fragment of human ACE2 consists of the sequence according to SEQ ID No. 2 and comprises a mutation at residues 619, 621, 625, 697, 702, 705, 708, 710 and 716, the numbering referring to SEQ ID No. 1. In one embodiment, the fragment of human ACE2 consists of the sequence according to SEQ ID No. 2 and comprises at least one of the following mutations: K619A, R621A, K625A, R697A, K702A, R705A, R708A, R710A and R716A, the numbering referring to SEQ ID No. 1. In one embodiment, the fragment of human ACE2 consists of the sequence according to SEQ ID No. 2 and comprises the following mutations: K619A, R621A, K625A, R697A, K702A, R705A, R708A, R710A and R716A, the numbering referring to SEQ ID No. 1.

In one embodiment, the fragment of human ACE2 consists of the sequence according to SEQ ID No. 3 and comprises a mutation at at least one residue selected from amino acids 619, 621, 625, 697, 702, 705, 708, 710 and 716, the numbering referring to SEQ ID No. 1. In one embodiment, the fragment of human ACE2 consists of the sequence according to SEQ ID No. 3 and comprises a mutation at residues 619, 621, 625, 697, 702, 705, 708, 710 and 716, the numbering referring to SEQ ID No. 1. In one embodiment, the fragment of human ACE2 consists of the sequence according to SEQ ID No. 3 and comprises at least one of the following mutations: K619A, R621A, K625A, R697A, K702A, R705A, R708A, R710A and R716A, the numbering referring to SEQ ID No. 1. In one embodiment, the fragment of human ACE2 consists of the sequence according to SEQ ID No. 3 and comprises the following mutations: K619A, R621A, K625A, R697A, K702A, R705A, R708A, R710A and R716A, the numbering referring to SEQ ID No. 1.

In one embodiment, the fragment of human ACE2 consists of the sequence according to SEQ ID No. 2 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID No. 1.

In one embodiment, the fragment of human ACE2 consists of the sequence according to SEQ ID No. 3 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID No. 1.

In one embodiment, the fragment of human ACE2 consists of the protein having the amino acid sequence according to SEQ ID No. 2 and comprises a R273A mutation, a L584A mutation and the following mutations: K619A, R621A, K625A, R697A, K702A, R705A, R708A, R710A and R716A, the numbering referring to the sequence according to SEQ ID No. 1.

In one embodiment, the fragment of human ACE2 consists of the protein having the amino acid sequence according to SEQ ID No. 3 and comprises a R273A mutation, a L584A mutation and the following mutations: K619A, R621A, K625A, R697A, K702A, R705A, R708A, R710A and R716A, the numbering referring to the sequence according to SEQ ID No. 1.

In one embodiment, the fragment of human ACE2 comprises or is identified by the sequence according to SEQ ID No. 2 and comprises a mutation at at least one residue selected from amino acids 697, 702, 705, 708, 710 and 716, the numbering referring to SEQ ID No. 1. In one embodiment, the fragment of human ACE2 comprises or is identified by the sequence according to SEQ ID No. 2 and comprises a mutation at residues 697, 702, 705, 708, 710 and 716, the numbering referring to SEQ ID No. 1. In one embodiment, the fragment of human ACE2 comprises or is identified by the sequence according to SEQ ID No. 2 and comprises at least one of the following mutations: R697A, K702A, R705A, R708A, R710A and R716A, the numbering referring to SEQ ID No. 1. In one embodiment, the fragment of human ACE2 comprises or is identified by the sequence according to SEQ ID No. 2 and comprises the following mutations: R697A, K702A, R705A, R708A, R710A and R716A, the numbering referring to SEQ ID No. 1.

In one embodiment, the fragment of human ACE2 comprises or is identified by the sequence according to SEQ ID No. 3 and comprises a mutation at at least one residue selected from amino acids 697, 702, 705, 708, 710 and 716, the numbering referring to SEQ ID No. 1. In one embodiment, the fragment of human ACE2 comprises or is identified by the sequence according to SEQ ID No. 3 and comprises a mutation at residues 697, 702, 705, 708, 710 and 716, the numbering referring to SEQ ID No. 1. In one embodiment, the fragment of human ACE2 comprises or is identified by the sequence according to SEQ ID No. 3 and comprises at least one of the following mutations: R697A, K702A, R705A, R708A, R710A and R716A, the numbering referring to SEQ ID No. 1. In one embodiment, the fragment of human ACE2 comprises or is identified by the sequence according to SEQ ID No. 3 and comprises the following mutations: R697A, K702A, R705A, R708A, R710A and R716A, the numbering referring to SEQ ID No. 1.

In one embodiment, the fragment of human ACE2 comprises or is identified by the sequence according to SEQ ID No. 2 and comprises a H374N mutation, a H378N mutation, a L584A mutation and the following mutations: R697A, K702A, R705A, R708A, R710A and R716A, the numbering referring to the sequence according to SEQ ID No. 1.

In one embodiment, the fragment of human ACE2 comprises or is identified by the sequence according to SEQ ID No. 3 and comprises a H374N mutation, a H378N mutation, a L584A mutation and the following mutations: R697A, K702A, R705A, R708A, R710A and R716A, the numbering referring to the sequence according to SEQ ID No. 1.

In one embodiment, the fragment of human ACE2 comprises or is identified by the sequence according to SEQ ID No. 2 and comprises a R273A mutation, a L584A mutation and the following mutations: R697A, K702A, R705A, R708A, R710A and R716A, the numbering referring to the sequence according to SEQ ID No. 1.

In one embodiment, the fragment of human ACE2 comprises or is identified by the sequence according to SEQ ID No. 3 and comprises a R273A mutation, a L584A mutation and the following mutations: R697A, K702A, R705A, R708A, R710A and R716A, the numbering referring to the sequence according to SEQ ID No. 1.

In one embodiment, the fragment of human ACE2 comprises or is identified by the sequence according to SEQ ID No. 2 and comprises a mutation at at least one residue selected from amino acids 619, 621, 625, 697, 702, 705, 708, 710 and 716, the numbering referring to SEQ ID No. 1. In one embodiment, the fragment of human ACE2 comprises or is identified by the sequence according to SEQ ID No. 2 and comprises a mutation at residues 619, 621, 625, 697, 702, 705, 708, 710 and 716, the numbering referring to SEQ ID No. 1. In one embodiment, the fragment of human ACE2 comprises or is identified by the sequence according to SEQ ID No. 2 and comprises at least one of the following mutations: K619A, R621A, K625A, R697A, K702A, R705A, R708A, R710A and R716A, the numbering referring to SEQ ID No. 1. In one embodiment, the fragment of human ACE2 comprises or is identified by the sequence according to SEQ ID No. 2 and comprises the following mutations: K619A, R621A, K625A, R697A, K702A, R705A, R708A, R710A and R716A, the numbering referring to SEQ ID No. 1.

In one embodiment, the fragment of human ACE2 comprises or is identified by the sequence according to SEQ ID No. 3 and comprises a mutation at at least one residue selected from amino acids 619, 621, 625, 697, 702, 705, 708, 710 and 716, the numbering referring to SEQ ID No. 1. In one embodiment, the fragment of human ACE2 comprises or is identified by the sequence according to SEQ ID No. 3 and comprises a mutation at residues 619, 621, 625, 697, 702, 705, 708, 710 and 716, the numbering referring to SEQ ID No. 1. In one embodiment, the fragment of human ACE2 comprises or is identified by the sequence according to SEQ ID No. 3 and comprises at least one of the following mutations: K619A, R621A, K625A, R697A, K702A, R705A, R708A, R710A and R716A, the numbering referring to SEQ ID No. 1. In one embodiment, the fragment of human ACE2 comprises or is identified by the sequence according to SEQ ID No. 3 and comprises the following mutations: K619A, R621A, K625A, R697A, K702A, R705A, R708A, R710A and R716A, the numbering referring to SEQ ID No. 1.

In one embodiment, the fragment of human ACE2 comprises or is identified by the sequence according to SEQ ID No. 2 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID No. 1.

In one embodiment, the fragment of human ACE2 comprises or is identified by the sequence according to SEQ ID No. 3 and comprises a H374N mutation, a H378N mutation, a L584A mutation and the following mutations: K619A, R621A, K625A, R697A, K702A, R705A, R708A, R710A and R716A, the numbering referring to the sequence according to SEQ ID No. 1.

In one embodiment, the fragment of human ACE2 comprises or is identified by the sequence according to SEQ ID No. 2 and comprises a R273A mutation, a L584A mutation and the following mutations: K619A, R621A, K625A, R697A, K702A, R705A, R708A, R710A and R716A, the numbering referring to the sequence according to SEQ ID No. 1.

In one embodiment, the fragment of human ACE2 comprises or is identified by the sequence according to SEQ ID No. 3 and comprises a R273A mutation, a L584A mutation and the following mutations: K619A, R621A, K625A, R697A, K702A, R705A, R708A, R710A and R716A, the numbering referring to the sequence according to SEQ ID No. 1.

Another variant of the fragment of human ACE2 may be a variant which provides an additional cysteine for the formation of disulfide bridges between two ACE2 molecules. The disulfide bridge increases the intrinsic stability of the fusion protein and may also have an effect on the binding of the fusion protein to its target. The additional cysteine may be provided by a substitution of serine 645 in the numbering of SEQ ID NO. 1 with cysteine.

Hence, in one embodiment, the variant of the fragment of human ACE2 comprises a S645C mutation, the numbering referring to SEQ ID No. 1.

In one embodiment, the fragment of human ACE2 consists of the sequence according to SEQ ID No. 2 and comprises a S645C mutation, the numbering referring to SEQ ID No. 1. In one embodiment, the variant of the fragment of human ACE2 consists of a protein having the amino acid sequence according to SEQ ID No. 28.

In one embodiment, the fragment of human ACE2 consists of the sequence according to SEQ ID No. 3 and comprises a S645C mutation, the numbering referring to SEQ ID No. 1. In one embodiment, the variant of the fragment of human ACE2 consists of a protein having the amino acid sequence according to SEQ ID No. 29.

In one embodiment, the fragment of human ACE2 consists of a protein having the amino acid sequence according to SEQ ID No. 2 and comprises a H374N mutation, a H378N mutation, and a S645C mutation, the numbering referring to SEQ ID No. 1. In one embodiment, the variant of the fragment of human ACE2 consists of a protein having the amino acid sequence according to SEQ ID No. 32.

In one embodiment, the fragment of human ACE2 consists of a protein having the amino acid sequence according to SEQ ID No. 3 and comprises a H374N mutation, a H378N mutation, and a S645C mutation, the numbering referring to SEQ ID No. 1. In one embodiment, the variant of the fragment of human ACE2 consists of a protein having the amino acid sequence according to SEQ ID No. 33.

In one embodiment, the fragment of human ACE2 consists of the sequence according to SEQ ID No. 2 and comprises a H374N mutation, a H378N mutation, a L584A mutation and a S645C mutation, the numbering referring to SEQ ID No. 1.

In one embodiment, the fragment of human ACE2 consists of the sequence according to SEQ ID No. 3 and comprises a H374N mutation, a H378N mutation, a L584A mutation and a S645C mutation, the numbering referring to SEQ ID No. 1.

In one embodiment, the fragment of human ACE2 consists of a protein having the amino acid sequence according to SEQ ID No. 2 and comprises a R273A mutation, and a S645C mutation, the numbering referring to SEQ ID No. 1. In one embodiment, the variant of the fragment of human ACE2 consists of a protein having the amino acid sequence according to SEQ ID No. 36.

In one embodiment, the fragment of human ACE2 consists of a protein having the amino acid sequence according to SEQ ID No. 3 and comprises a R273A mutation and a S645C mutation, the numbering referring to SEQ ID No. 1. In one embodiment, the variant of the fragment of human ACE2 consists of a protein having the amino acid sequence according to SEQ ID No. 37.

In one embodiment, the fragment of human ACE2 consists of a protein having the amino acid sequence according to SEQ ID No. 2 and comprises a R273A mutation, a L584A mutation and a S645C mutation, the numbering referring to SEQ ID No. 1.

In one embodiment, the fragment of human ACE2 consists of a protein having the amino acid sequence according to SEQ ID No. 3 and comprises a R273A mutation, a L584A mutation and a S645C mutation, the numbering referring to SEQ ID No. 1.

In one embodiment, the fragment of human ACE2 comprises or is identified by the sequence according to SEQ ID No. 2 and comprises a S645C mutation, the numbering referring to SEQ ID No. 1.

In one embodiment, the fragment of human ACE2 comprises or is identified by the sequence according to SEQ ID No. 3 and comprises a S645C mutation, the numbering referring to SEQ ID No. 1.

In one embodiment, the fragment of human ACE2 comprises or is identified by the sequence according to SEQ ID No. 2 and comprises a H374N mutation, a H378N mutation and a S645C mutation, the numbering referring to SEQ ID No. 1.

In one embodiment, the fragment of human ACE2 comprises or is identified by the sequence according to SEQ ID No. 3 and comprises a H374N mutation, a H378N mutation and a S645C mutation, the numbering referring to SEQ ID No. 1.

In one embodiment, the fragment of human ACE2 comprises or is identified by the sequence according to SEQ ID No. 2 and comprises a H374N mutation, a H378N mutation, a L584A mutation and a S645C mutation, the numbering referring to SEQ ID No. 1.

In one embodiment, the fragment of human ACE2 comprises or is identified by the sequence according to SEQ ID No. 3 and comprises a H374N mutation, a H378N mutation, a L584A mutation and a S645C mutation, the numbering referring to SEQ ID No. 1.

In one embodiment, the fragment of human ACE2 comprises or is identified by the sequence according to SEQ ID No. 2 and comprises a R273A mutation and a S645C mutation, the numbering referring to SEQ ID No. 1.

In one embodiment, the fragment of human ACE2 comprises or is identified by the sequence according to SEQ ID No. 3 and comprises a R273A and a S645C mutation, the numbering referring to SEQ ID No. 1.

In one embodiment, the fragment of human ACE2 comprises or is identified by the sequence according to SEQ ID No. 2 and comprises a R273A mutation, a L584A mutation and a S645C mutation, the numbering referring to SEQ ID No. 1.

In one embodiment, the fragment of human ACE2 comprises or is identified by the sequence according to SEQ ID No. 3 and comprises a R273A mutation, a L584A mutation and a S645C mutation, the numbering referring to SEQ ID No. 1.

Another variant of the fragment of human ACE2 may be a variant which inhibits dimerization. Hence, in one embodiment the variant of the fragment of human ACE2 comprises a mutation at amino acid Q139, the numbering referring to SEQ ID No. 1. In one embodiment the variant of the fragment of human ACE2 comprises a Q139A mutation, the numbering referring to SEQ ID No. 1.

In one embodiment, the fragment of human ACE2 consists of the sequence according to SEQ ID No. 2 and comprises a Q139A mutation, the numbering referring to SEQ ID No. 1.

In one embodiment, the fragment of human ACE2 consists of the sequence according to SEQ ID No. 3 and comprises a Q139A mutation, the numbering referring to SEQ ID No. 1.

In one embodiment, the fragment of human ACE2 consists of the protein having the amino acid sequence according to SEQ ID No. 2 and comprises a H374N mutation, a H378N mutation and a Q139A mutation, the numbering referring to SEQ ID No. 1.

In one embodiment, the fragment of human ACE2 consists of the protein having the amino acid sequence according to SEQ ID No. 3 and comprises a H374N mutation, a H378N mutation and a Q139A mutation, the numbering referring to SEQ ID No. 1.

In one embodiment, the fragment of human ACE2 consists of the sequence according to SEQ ID No. 2 and comprises a H374N mutation, a H378N mutation, a L584A mutation and a Q139A mutation, the numbering referring to SEQ ID No. 1.

In one embodiment, the fragment of human ACE2 consists of the sequence according to SEQ ID No. 3 and comprises a H374N mutation, a H378N mutation, a L584A mutation and a Q139A mutation, the numbering referring to SEQ ID No. 1.

In one embodiment, the fragment of human ACE2 consists of the protein having the amino acid sequence according to SEQ ID No. 2 and comprises a R273A mutation and a Q139A mutation, the numbering referring to SEQ ID No. 1.

In one embodiment, the fragment of human ACE2 consists of the protein having the amino acid sequence according to SEQ ID No. 3 and comprises a R273A mutation and a Q139A mutation, the numbering referring to SEQ ID No. 1.

In one embodiment, the fragment of human ACE2 consists of the protein having the amino acid sequence according to SEQ ID No. 2 and comprises a R273A mutation, a L584A mutation and a Q139A mutation, the numbering referring to SEQ ID No. 1.

In one embodiment, the fragment of human ACE2 consists of the protein having the amino acid sequence according to SEQ ID No. 3 and comprises a R273A mutation, a L584A mutation and a Q139A mutation, the numbering referring to SEQ ID No. 1.

In one embodiment, the fragment of human ACE2 consists of the sequence according to SEQ ID No. 14 and comprises a Q139A mutation, the numbering referring to SEQ ID No. 1.

In one embodiment, the fragment of human ACE2 consists of the sequence according to SEQ ID No. 14 and comprises a H374N mutation, a H378N mutation, a L584A mutation and a Q139A mutation, the numbering referring to SEQ ID No. 1.

In one embodiment, the fragment of human ACE2 consists of the protein having the amino acid sequence according to SEQ ID No. 14 and comprises a R273A mutation, a L584A mutation and a Q139A mutation, the numbering referring to SEQ ID No. 1.

In one embodiment, the fragment of human ACE2 comprises or is identified by the sequence according to SEQ ID No. 2 and comprises a Q139A mutation, the numbering referring to SEQ ID No. 1.

In one embodiment, the fragment of human ACE2 comprises or is identified by the sequence according to SEQ ID No. 3 and comprises a Q139A mutation, the numbering referring to SEQ ID No. 1.

In one embodiment, the fragment of human ACE2 comprises or is identified by the sequence according to SEQ ID No. 2 and comprises a H374N mutation, a H378N mutation and a Q139A mutation, the numbering referring to SEQ ID No. 1.

In one embodiment, the fragment of human ACE2 comprises or is identified by the sequence according to SEQ ID No. 3 and comprises a H374N mutation, a H378N mutation and a Q139A mutation, the numbering referring to SEQ ID No. 1.

In one embodiment, the fragment of human ACE2 comprises or is identified by the sequence according to SEQ ID No. 2 and comprises a H374N mutation, a H378N mutation, a L584A mutation and a Q139A mutation, the numbering referring to SEQ ID No. 1.

In one embodiment, the fragment of human ACE2 comprises or is identified by the sequence according to SEQ ID No. 3 and comprises a H374N mutation, a H378N mutation, a L584A mutation and a Q139A mutation, the numbering referring to SEQ ID No. 1.

In one embodiment, the fragment of human ACE2 comprises or is identified by the sequence according to SEQ ID No. 2 and comprises a R273A mutation and a Q139A mutation, the numbering referring to SEQ ID No. 1.

In one embodiment, the fragment of human ACE2 comprises or is identified by the sequence according to SEQ ID No. 3 and comprises a R273A mutation and a Q139A mutation, the numbering referring to SEQ ID No. 1.

In one embodiment, the fragment of human ACE2 comprises or is identified by the sequence according to SEQ ID No. 2 and comprises a R273A mutation, a L584A mutation and a Q139A mutation, the numbering referring to SEQ ID No. 1.

25 In one embodiment, the fragment of human ACE2 comprises or is identified by the sequence according to SEQ ID No. 3 and comprises a R273A mutation, a L584A mutation and a Q139A mutation, the numbering referring to SEQ ID No. 1.

In one embodiment, the fragment of human ACE2 comprises or is identified by the sequence according to SEQ ID No. 14 and comprises a Q139A mutation, the numbering referring to SEQ ID No. 1.

In one embodiment, the fragment of human ACE2 comprises or is identified by the sequence according to SEQ ID No. 14 and comprises a H374N mutation, a H378N mutation and a Q139A mutation, the numbering referring to SEQ ID No. 1.

In one embodiment, the fragment of human ACE2 comprises or is identified by the sequence according to SEQ ID No. 14 and comprises a H374N mutation, a H378N mutation, a L584A mutation and a Q139A mutation, the numbering referring to SEQ ID No. 1.

In one embodiment, the fragment of human ACE2 comprises or is identified by the sequence according to SEQ ID No. 14 and comprises a R273A mutation and a Q139A mutation, the numbering referring to SEQ ID No. 1.

In one embodiment, the fragment of human ACE2 comprises or is identified by the sequence according to SEQ ID No. 14 and comprises a R273A mutation, a L584A mutation and a Q139A mutation, the numbering referring to SEQ ID No. 1.

In one embodiment, the second part of the fusion protein of the present invention comprises the Fc portion of human IgG. The Fc portion of human IgG may be the Fc portion of IgG1, IgG2, IgG3 or IgG4.

In one embodiment, the second part of the fusion protein of the present invention comprises the Fc portion of human IgG4. The Fc portion of human IgG4 comprises the CH2 and CH3 domains of human IgG4 linked together to form the Fc portion. In a full-length human IgG4 antibody the Fc portion is connected to the Fab fragment through a hinge region. The Fab fragment comprises the heavy chain variable region and the CH1 domain. Preferably, the Fc portion of human IgG4 used in the fusion protein of the present invention has the sequence according to SEQ ID No. 5.

Since the IgG4 subclass of antibodies has only a partial affinity for Fc gamma receptor and does not activate complement (see Muhammed (2020) Immunome Res. 16(1): 173), it does not activate the immune system to the same extent as the IgG1 subclass of antibodies. Consequently, the cytokine expression is stimulated to a lower extent and the risk for a cytokine storm is reduced. The IgG4 subclass of antibodies is able to bind to FcRn.

“A variant of the Fc portion of human IgG4” refers to the Fc portion of human IgG4 which has one or more amino acid substitutions compared to the wild-type Fc portion of human IgG4 according to SEQ ID No. 5. In one embodiment, the variant of the Fc portion of human IgG4 has one to twelve, one to eleven, one to ten, one to nine, one to eight, one to seven, one to six, one to five, one to four, one to three, one or two amino acid substitutions compared to the wild-type Fc portion of human IgG4 according to SEQ ID No. 5. In one embodiment, the variant of the Fc portion of human IgG4 has one, two, three, four, five, six, seven, eight, nine, ten, eleven or twelve amino acid substitutions compared to the wild-type Fc portion of human IgG4 according to SEQ ID No. 5. In one embodiment, the one or more amino acid substitutions lead to decreased effector functions compared to the wild-type Fc portion of human IgG4 according to SEQ ID No. 5. In one embodiment, the one or more amino acid substitutions lead to an increased half-life compared to the wild-type Fc portion of human IgG4 according to SEQ ID No. 5. In one embodiment, the one or more amino acid substitutions lead to decreased effector functions compared to the wild-type Fc portion of human IgG4 according to SEQ ID No. 5 and to an increased half-life compared to the wild-type Fc portion of human IgG4 according to SEQ ID No. 5.

In one embodiment, the one or more amino acid substitutions do not produce the wild-type Fc portion of IgG1 according to SEQ ID No. 16. In one embodiment, the one or more amino acid substitutions do not impart upon the altered IgG4 Fc portion the effector functions of wild-type IgG1.

Preferably, the decreased effector functions comprise a decreased complement-dependent cytotoxicity (CDC). More preferably, the CDC is decreased by at least two-fold, at least three-fold, at least four-fold or at least five-fold compared to the CDC of the wild-type Fc portion of human IgG4 according to SEQ ID No. 5. Methods to determine and quantify CDC are well-known to the skilled person. In general, CDC can be determined by incubating the Fc portion fused to an antigen-binding portion with suitable target cells and complement and detecting the cell death of the target cells. Complement recruitment can be analyzed with a C1q binding assay using ELISA plates (see, e.g., Schlothauer et al. (2016) Protein Eng. Des. Sel. 29(10): 457-466).

In one embodiment, the variant of the Fc portion of human IgG4 comprises at least one amino acid substitution at an amino acid residue selected from F3, L4, G6, P7, F12, V33, N66 and P98 of the sequence according to SEQ ID No. 5. These amino acid residues correspond to amino acid residues F234, L235, G237, P238, F243, V264, N297 and P329 of full-length human IgG4. It was shown that amino acid substitutions at these residues lead to a reduced effector function (WO 94/28027; WO 94/29351; WO 95/26403; WO 2011/066501; WO 2011/149999; WO 2012/130831; Wang et al. (2018) Protein Cell. 9(1): 63-73).

In one embodiment, the variant of the Fc portion of human IgG4 comprises the amino acid substitution L4E/A in the sequence according to SEQ ID No. 5 which corresponds to the amino acid substitution L235E/A in the amino acid sequence of full-length human IgG4. This variant has a reduced effector function, in particular reduced CDC.

In one embodiment, the variant of the Fc portion of human IgG4 comprises the amino acid substitutions F3A and L4A in the sequence according to SEQ ID No. 5 which correspond to the amino acid substitutions F234A and L235A in the amino acid sequence of full-length human IgG4. This variant has a reduced effector function, in particular reduced CDC.

In one embodiment, the variant of the Fc portion of human IgG4 comprises the amino acid substitutions F3A, L4E, G6A and P7S in the sequence according to SEQ ID No. 5 which correspond to the amino acid substitutions F234A, L235E, G237A and P238S in the amino acid sequence of full-length human IgG4. This variant has a reduced effector function, in particular reduced CDC.

In one embodiment, the variant of the Fc portion of human IgG4 comprises the amino acid substitutions F12A and V33A in the sequence according to SEQ ID No. 5 which correspond to the amino acid substitutions F243A and V264A in the amino acid sequence of full-length human IgG4. This variant has a reduced effector function, in particular reduced CDC.

In one embodiment, the variant of the Fc portion of human IgG4 comprises the amino acid substitutions L4E and P98G in the sequence according to SEQ ID No. 5 which correspond to the amino acid substitutions L235E and P329G in the amino acid sequence of full-length human IgG4. This variant has a reduced effector function, in particular reduced CDC.

In one embodiment, the variant of the Fc portion of human IgG4 comprises the amino acid substitution N66A/Q/G in the sequence according to SEQ ID No. 5 which corresponds to the amino acid substitution N297A/Q/G in the amino acid sequence of full-length human IgG4. This variant has a reduced effector function, in particular reduced CDC.

In one embodiment, the variant of the Fc portion of human IgG4 comprises at least one amino acid substitution at an amino acid residue selected from T250, M252, S254, T256, E258, K288, T307, V308, Q311, V427, M428, H433, N434 and H435 of full-length human IgG4. These amino acid residues correspond to amino acid residues T19, M21, S23, T25, E27, K57, T76, V77, Q80, V196, M197, H202, N203 and H204 of the sequence according to SEQ ID No. 5. It was shown that these amino acid substitutions lead to an increased half-life of the Fc-containing protein (WO 00/42072; WO 02/060919; WO 2004/035752; WO 2006/053301; WO 2009/058492; WO 2009/086320; US 2010/0204454; GB 2013/02878; WO 2013/163630; US 2019/0010243). The half-life of an antibody or Fc fusion protein can be determined by measuring the antibody or Fc fusion protein concentration in the serum at different time-points after administration of the antibody or Fc fusion protein and calculating the half-life therefrom.

In one embodiment, the variant of the Fc portion of human IgG4 comprises the amino acid substitutions M21Y, S23T and T25E in the sequence according to SEQ ID No. 5 which corresponds to the amino acid substitutions M252Y, S254T and T256E in the amino acid sequence of full-length human IgG4. In one embodiment, the variant of the Fc portion of human IgG4 has the amino acid sequence according to SEQ ID No. 44. This variant has an increased half-life.

In one embodiment, the variant of the Fc portion of human IgG4 comprises the amino acid substitutions T25D and T76Q in the sequence according to SEQ ID No. 5 which corresponds to the amino acid substitutions T256D and T307Q in the amino acid sequence of full-length human IgG4. In one embodiment, the variant of the Fc portion of human IgG4 has the amino acid sequence according to SEQ ID No. 60. This variant has an increased half-life and an enhanced binding to FcRn (Mackness et al. (2019) MABS 11(7): 1276-1288).

In one embodiment, the variant of the Fc portion of human IgG4 comprises the amino acid substitutions T19Q/E and M197L/F in the sequence according to SEQ ID No. 5 which corresponds to the amino acid substitutions T250Q/E and M428L/F in the amino acid sequence of full-length human IgG4. This variant has an increased half-life.

In one embodiment, the variant of the Fc portion of human IgG4 comprises the amino acid substitutions N2035 and V77W/Y/F in the sequence according to SEQ ID No. 5 which corresponds to the amino acid substitutions N434S and V308W/Y/F in the amino acid sequence of full-length human IgG4. This variant has an increased half-life.

In one embodiment, the variant of the Fc portion of human IgG4 comprises the amino acid substitutions M21Y and M197L in the sequence according to SEQ ID No. 5 which corresponds to the amino acid substitutions M252Y and M428L in the amino acid sequence of full-length human IgG4. This variant has an increased half-life.

In one embodiment, the variant of the Fc portion of human IgG4 comprises the amino acid substitutions T76Q and N2035 in the sequence according to SEQ ID No. 5 which corresponds to the amino acid substitutions T307Q and N434S in the amino acid sequence of full-length human IgG4. This variant has an increased half-life.

In one embodiment, the variant of the Fc portion of human IgG4 comprises the amino acid substitutions M197L and V77F in the sequence according to SEQ ID No. 5 which corresponds to the amino acid substitutions M428L and V308F in the amino acid sequence of full-length human IgG4. This variant has an increased half-life.

In one embodiment, the variant of the Fc portion of human IgG4 comprises the amino acid substitutions Q80V and N2035 in the sequence according to SEQ ID No. 5 which corresponds to the amino acid substitutions Q311V and N434S in the amino acid sequence of full-length human IgG4. This variant has an increased half-life.

In one embodiment, the variant of the Fc portion of human IgG4 comprises the amino acid substitutions H202K and N203F in the sequence according to SEQ ID No. 5 which corresponds to the amino acid substitutions H433K and N434F in the amino acid sequence of full-length human IgG4. This variant has an increased half-life.

In one embodiment, the variant of the Fc portion of human IgG4 comprises the amino acid substitutions E27F and V196T in the sequence according to SEQ ID No. 5 which corresponds to the amino acid substitutions E258F and V427T in the amino acid sequence of full-length human IgG4. This variant has an increased half-life.

In one embodiment, the variant of the Fc portion of human IgG4 comprises the amino acid substitutions K57E and H204K in the sequence according to SEQ ID No. 5 which corresponds to the amino acid substitutions K288E and H435K in the amino acid sequence of full-length human IgG4. This variant has an increased half-life.

In one embodiment, the variant of the Fc portion of human IgG4 comprises the amino acid substitution R178K in the sequence according to SEQ ID No. 5 which corresponds to the amino acid substitution R409K in the amino acid sequence of full-length human IgG4. This variant prevents acid-induced aggregation of human IgG4 (see Namisaki et al. (2020) PloS ONE 15(3): e0229027).

In one embodiment, the variant of the Fc portion of human IgG4 does not comprise an amino acid substitution at one or more of positions 37, 43, 65, 96, 99, 100, 124, 125, 127, 187 and 214 in the sequence according to SEQ ID No. 5. In one embodiment, the variant of the Fc portion of human IgG4 does not comprise one or more of the amino acid substitutions Q37H, Q43K, F65Y, G96A, S99A, S100P, Q124R, E125D, M127L, R178K, E187Q and L214P. In one embodiment, the variant of the Fc portion of human IgG4 does not comprise any amino acid substitution at any of the positions 37, 43, 65, 96, 99, 100, 124, 125, 127, 187 and 214 in the sequence according to SEQ ID No. 5. In one embodiment, the variant of the Fc portion of human IgG4 does not comprise any of the amino acid substitutions Q37H, Q43K, F65Y, G96A, S99A, S100P, Q124R, E125D, M127L, R178K, E187Q and L214P.

In one embodiment, the second part of the fusion protein of the present invention comprises the Fc portion of human IgG1 or a variant thereof. The Fc portion of human IgG1 comprises the CH2 and CH3 domains of human IgG1 linked together to form the Fc portion. In a full-length human IgG1 antibody the Fc portion is connected to the Fab fragment through a hinge region. The Fab fragment comprises the heavy chain variable region and the CH1 domain. Preferably, the Fc portion of human IgG1 used in the fusion protein of the present invention has the sequence according to SEQ ID No. 16.

“A variant of the Fc portion of human IgG1” refers to the Fc portion of human IgG1 which has one or more amino acid substitutions compared to the wild-type Fc portion of human IgG1 according to SEQ ID No. 16. In one embodiment, the one or more amino acid substitutions lead to decreased effector functions compared to the wild-type Fc portion of human IgG1 according to SEQ ID No. 16.

Preferably, the decreased effector functions comprise a decreased complement-dependent cytotoxicity (CDC). More preferably, the CDC is decreased by at least two-fold, at least three-fold, at least four-fold or at least five-fold compared to the CDC of the wild-type Fc portion of human IgG1 according to SEQ ID No. 16. Methods to determine and quantify CDC are well-known to the skilled person and have been described above.

In one embodiment, the variant of the Fc portion of human IgG1 comprises at least one amino acid substitution at an amino acid residue selected from L3, L4 and P98 of the sequence according to SEQ ID No. 16. These amino acid residues correspond to amino acid residues L234, L235, and P329 of full-length human IgG1.

In one embodiment, the variant of the Fc portion of human IgG1 comprises the amino acid substitution L4E/A in the sequence according to SEQ ID No. 16 which corresponds to the amino acid substitution L235E/A in the amino acid sequence of full-length human IgG1. This variant has a reduced effector function, in particular reduced CDC.

In one embodiment, the variant of the Fc portion of human IgG1 comprises the amino acid substitutions L3A and L4A in the sequence according to SEQ ID No. 16 which correspond to the amino acid substitutions L234A and L235A in the amino acid sequence of full-length human IgG1. This variant has a reduced effector function, in particular reduced CDC.

In one embodiment, the variant of the Fc portion of human IgG1 comprises the amino acid substitutions L3A, L4A, P98G in the sequence according to SEQ ID No. 16 which correspond to the amino acid substitutions L234A, L235A and P329G in the amino acid sequence of full-length human IgG1. This variant has a reduced effector function, in particular reduced CDC.

In one embodiment, the variant of the Fc portion of human IgG1 comprises the amino acid substitutions L4A and P98G in the sequence according to SEQ ID No. 16 which correspond to the amino acid substitutions L235A and P329G in the amino acid sequence of full-length human IgG1. This variant has a reduced effector function, in particular reduced CDC.

In one embodiment, the variant of the Fc portion of human IgG1 comprises the amino acid substitutions M21Y, S23T and T25E in the sequence according to SEQ ID No. 16 which corresponds to the amino acid substitutions M252Y, S254T and T256E in the amino acid sequence of full-length human IgG1. In one embodiment, the variant of the Fc portion of human IgG1 has the amino acid sequence according to SEQ ID No. 19. This variant has an increased half-life and an enhanced binding to FcRn.

In one embodiment, the variant of the Fc portion of human IgG1 comprises the amino acid substitutions T25D and T76Q in the sequence according to SEQ ID No. 16 which corresponds to the amino acid substitutions T256D and T307Q in the amino acid sequence of full-length human IgG1. In one embodiment, the variant of the Fc portion of human IgG1 has the amino acid sequence according to SEQ ID No. 59. This variant has an increased half-life and an enhanced binding to FcRn (Mackness et al. (2019) MABS 11(7): 1276-1288).

In one embodiment, the second part of the fusion protein of the present invention comprises the Fc portion of human IgG2 or IgG3 or a variant of the Fc portion of human IgG1, IgG2 or IgG3 and has reduced binding to FcγRIIIa compared to a fusion protein comprising the same first part and a second part comprising the Fc portion of wild-type human IgG1. The binding to FcγRIIIa is decreased by at least two-fold, at least three-fold, at least four-fold, at least five-fold or at least 10-fold compared to the binding of a fusion protein comprising the same first part and a second part comprising the Fc portion of wild-type human IgG1 according to SEQ ID No. 16.

In one embodiment, the second part of the fusion protein of the present invention comprises the Fc portion of human IgG2 or IgG3 or a variant of the Fc portion of human IgG1, IgG2 or IgG3 and has reduced binding to FcγRIIIa and essentially the same binding to FcRn compared to a fusion protein comprising the same first part and a second part comprising the Fc portion of wild-type human IgG1. The term “essentially the same binding to FcRn” means that the binding of the fusion protein comprising the Fc portion of human IgG2 or IgG3 or a variant of the Fc portion of human IgG1, IgG2 or IgG3 to FcRn differs by not more than 20% or not more than 15%, preferably not more than 10% or not more than 5%, more preferably not more than 3% or not more than 2% and most preferably not more than 1% from the binding of a fusion protein comprising the same first part and a second part comprising the Fc portion of wild-type human IgG1 according to SEQ ID No. 16.

The binding of fusion proteins to FcγRIIIa or FcRn can be determined by surface plasmon resonance as described in the examples herein.

In one embodiment, the first and the second part of the fusion protein of the present invention are linked by a linking sequence. The linking sequence is a short amino acid sequence which does not have a function on its own and which does not affect the folding of the fusion protein. In one embodiment, the linking sequence comprises eight to twenty amino acids, preferably 10 to 18 amino acids, more preferably 11 to 17 amino acids or 12 to 16 amino acids and most preferably 13 amino acids.

In one embodiment, the linking sequence consists of small amino acids selected from glycine and serine. An overview of linking sequences is provided in Chen et al. (2013) Adv. Drug Deliv. Rev. 65(10): 1357-1369.

In one embodiment, if the second part of the fusion protein is the Fc portion of human IgG4 or a variant thereof, the linking sequence consists of the hinge region of human IgG4. In one embodiment, the linking sequence consists of the sequence according to SEQ ID No. 4. In the sequence according to SEQ ID No. 4 the serine at residue 10 of the wild-type hinge region of IgG4 (corresponding to serine 228 of full-length IgG4) has been replaced with proline, leading to a reduction in the exchange of IgG half-molecules. It is known that IgG4 antibodies can undergo Fab-arm exchange, leading to the combination of two distinct Fab arms and creating new bispecific antibody molecules (see, e.g., Aalberse et al. (2009) Clin. Exp. Allergy 39(4): 469-477). This Fab-arm exchange can be prevented by a mutation of serine 228 of the Fc region to proline (S228P; see Silva et al. (2015) J. Biol. Chem. 290: 5462-5469) which is located in the hinge region of IgG4. Further, the use of a short linker sequence increases the stability of the fusion protein and lowers the accessibility of the fusion protein to proteases.

In one embodiment, if the second part of the fusion protein is the Fc portion of human IgG1 or a variant thereof, the linking sequence consists of the hinge region of human IgG1. In one embodiment, the linking sequence consists of the sequence according to SEQ ID No. 15.

In a particular embodiment, the fusion protein of the present invention has the amino acid sequence according to SEQ ID No. 20 which comprises amino acids 18 to 732 of human ACE2 (SEQ ID No. 2), the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 21 which comprises amino acids 18 to 740 of human ACE2 (SEQ ID No. 3), the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 22 which comprises amino acids 18 to 732 of human ACE2 (SEQ ID No. 2) with a H374N and H378N mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 23 which comprises amino acids 18 to 740 of human ACE2 (SEQ ID No. 3) with a H374N and H378N mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 26 which comprises amino acids 18 to 732 of human ACE2 (SEQ ID No. 2) with a R273A mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 27 which comprises amino acids 18 to 740 of human ACE2 (SEQ ID No. 3) with a R273A mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 30 which comprises amino acids 18 to 732 of human ACE2 (SEQ ID No. 2) with a S645C mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 31 which comprises amino acids 18 to 740 of human ACE2 (SEQ ID No. 3) with a S645C mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 34 which comprises amino acids 18 to 732 of human ACE2 (SEQ ID No. 2) with a S645C mutation, a H374N mutation and a H378N mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 35 which comprises amino acids 18 to 740 of human ACE2 (SEQ ID No. 3) with a S645C mutation, a H374N mutation and a H378N mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 38 which comprises amino acids 18 to 732 of human ACE2 (SEQ ID No. 2) with a S645C mutation and a R273A mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 39 which comprises amino acids 18 to 740 of human ACE2 (SEQ ID No. 3) with a S645C mutation and a R273A mutation, the numbering referring to SEQ ID No. 1, the linking sequence according to SEQ ID No. 15 and the variant of the Fc portion of human IgG1 according to SEQ ID No. 19.

In a particular embodiment, the fusion protein of the present invention has the amino acid sequence according to SEQ ID No. 40 which comprises amino acids 18 to 732 of human ACE2 (SEQ ID No. 2) with a S645C mutation, the numbering referring to SEQ ID No. 1, the linking sequence according to SEQ ID No. 15 and the Fc portion of human IgG1 according to SEQ ID No. 16.

In a particular embodiment, the fusion protein of the present invention has the amino acid sequence according to SEQ ID No. 41 which comprises amino acids 18 to 740 of human ACE2 (SEQ ID No. 3) with a S645C mutation, the numbering referring to SEQ ID No. 1, the linking sequence according to SEQ ID No. 15 and the Fc portion of human IgG1 according to SEQ ID No. 16.

In a particular embodiment, the fusion protein of the present invention has the amino acid sequence according to SEQ ID No. 42 which comprises amino acids 18 to 732 of human ACE2 (SEQ ID No. 2) with a S645C mutation, the numbering referring to SEQ ID No. 1, the linking sequence according to SEQ ID No. 4 and the Fc portion of human IgG4 according to SEQ ID No. 5.

In a particular embodiment, the fusion protein of the present invention has the amino acid sequence according to SEQ ID No. 43 which comprises amino acids 18 to 740 of human ACE2 (SEQ ID No. 3) with a S645C mutation, the numbering referring to SEQ ID No. 1, the linking sequence according to SEQ ID No. 4 and the Fc portion of human IgG4 according to SEQ ID No. 5.

In a particular embodiment, the fusion protein of the present invention has the amino acid sequence according to SEQ ID No. 45 which comprises amino acids 18 to 732 of human ACE2 (SEQ ID No. 2) with a S645C mutation, the numbering referring to SEQ ID No. 1, the linking sequence according to SEQ ID No. 4 and the variant of the Fc portion of human IgG4 according to SEQ ID No. 44.

In a particular embodiment, the fusion protein of the present invention has the amino acid sequence according to SEQ ID No. 46 which comprises amino acids 18 to 740 of human ACE2 (SEQ ID No. 3) with a S645C mutation, the numbering referring to SEQ ID No. 1, the linking sequence according to SEQ ID No. 4 and the variant of Fc portion of human IgG4 according to SEQ ID No. 44.

In a particular embodiment, the fusion protein of the present invention has the amino acid sequence according to SEQ ID No. 47 which comprises amino acids 18 to 732 of human ACE2 (SEQ ID No. 2) with a S645C mutation, a H374N mutation and a H378N mutation, the numbering referring to SEQ ID No. 1, the linking sequence according to SEQ ID No. 15 and the Fc portion of human IgG1 according to SEQ ID No. 16.

In a particular embodiment, the fusion protein of the present invention has the amino acid sequence according to SEQ ID No. 48 which comprises amino acids 18 to 740 of human ACE2 (SEQ ID No. 3) with a S645C mutation, a H374N mutation and a H378N mutation, the numbering referring to SEQ ID No. 1, the linking sequence according to SEQ ID No. 15 and the Fc portion of human IgG1 according to SEQ ID No. 16.

In a particular embodiment, the fusion protein of the present invention has the amino acid sequence according to SEQ ID No. 49 which comprises amino acids 18 to 732 of human ACE2 (SEQ ID No. 2) with a S645C mutation and a R273A mutation, the numbering referring to SEQ ID No. 1, the linking sequence according to SEQ ID No. 15 and the Fc portion of human IgG1 according to SEQ ID No. 16.

In a particular embodiment, the fusion protein of the present invention has the amino acid sequence according to SEQ ID No. 50 which comprises amino acids 18 to 740 of human ACE2 (SEQ ID No. 3) with a S645C mutation and a a R273A mutation, the numbering referring to SEQ ID No. 1, the linking sequence according to SEQ ID No. 15 and the Fc portion of human IgG1 according to SEQ ID No. 16.

In a particular embodiment, the fusion protein of the present invention has the amino acid sequence according to SEQ ID No. 51 which comprises amino acids 18 to 732 of human ACE2 (SEQ ID No. 2) with a S645C mutation, a H374N mutation and a H378N mutation, the numbering referring to SEQ ID No. 1, the linking sequence according to SEQ ID No. 4 and the Fc portion of human IgG4 according to SEQ ID No. 5.

In a particular embodiment, the fusion protein of the present invention has the amino acid sequence according to SEQ ID No. 52 which comprises amino acids 18 to 740 of human ACE2 (SEQ ID No. 3) with a S645C mutation, a H374N mutation and a H378N mutation, the numbering referring to SEQ ID No. 1, the linking sequence according to SEQ ID No. 4 and the Fc portion of human IgG4 according to SEQ ID No. 5.

In a particular embodiment, the fusion protein of the present invention has the amino acid sequence according to SEQ ID No. 53 which comprises amino acids 18 to 732 of human ACE2 (SEQ ID No. 2) with a S645C mutation and a R273A mutation, the numbering referring to SEQ ID No. 1, the linking sequence according to SEQ ID No. 4 and the Fc portion of human IgG4 according to SEQ ID No. 5.

In a particular embodiment, the fusion protein of the present invention has the amino acid sequence according to SEQ ID No. 54 which comprises amino acids 18 to 740 of human ACE2 (SEQ ID No. 3) with a S645C mutation and a a R273A mutation, the numbering referring to SEQ ID No. 1, the linking sequence according to SEQ ID No. 4 and the Fc portion of human IgG4 according to SEQ ID No. 5.

In a particular embodiment, the fusion protein of the present invention has the amino acid sequence according to SEQ ID No. 55 which comprises amino acids 18 to 732 of human ACE2 (SEQ ID No. 2) with a S645C mutation, a H374N mutation and a H378N mutation, the numbering referring to SEQ ID No. 1, the linking sequence according to SEQ ID No. 4 and the variant of the Fc portion of human IgG4 according to SEQ ID No. 44.

In a particular embodiment, the fusion protein of the present invention has the amino acid sequence according to SEQ ID No. 56 which comprises amino acids 18 to 740 of human ACE2 (SEQ ID No. 3) with a S645C mutation, a H374N mutation and a H378N mutation, the numbering referring to SEQ ID No. 1, the linking sequence according to SEQ ID No. 4 and the variant of the Fc portion of human IgG4 according to SEQ ID No. 44.

In a particular embodiment, the fusion protein of the present invention has the amino acid sequence according to SEQ ID No. 57 which comprises amino acids 18 to 732 of human ACE2 (SEQ ID No. 2) with a S645C mutation and a R273A mutation, the numbering referring to SEQ ID No. 1, the linking sequence according to SEQ ID No. 4 and the variant of the Fc portion of human IgG4 according to SEQ ID No. 44.

In a particular embodiment, the fusion protein of the present invention has the amino acid sequence according to SEQ ID No. 58 which comprises amino acids 18 to 740 of human ACE2 (SEQ ID No. 3) with a S645C mutation and a a R273A mutation, the numbering referring to SEQ ID No. 1, the linking sequence according to SEQ ID No. 4 and the variant of the Fc portion of human IgG4 according to SEQ ID No. 44.

In a particular embodiment, the fusion protein of the present invention has the amino acid sequence according to SEQ ID No. 61 which comprises amino acids 18 to 732 of human ACE2 (SEQ ID No. 2) with a S645C mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 62 which comprises amino acids 18 to 740 of human ACE2 (SEQ ID No. 3) with a S645C mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 63 which comprises amino acids 18 to 732 of human ACE2 (SEQ ID No. 2) with a S645C mutation, a H374N mutation and a H378N mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 64 which comprises amino acids 18 to 740 of human ACE2 (SEQ ID No. 3) with a S645C mutation, a H374N mutation and a H378N mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 65 which comprises amino acids 18 to 732 of human ACE2 (SEQ ID No. 2) with a S645C mutation and a R273A mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 66 which comprises amino acids 18 to 740 of human ACE2 (SEQ ID No. 3) with a S645C mutation and a R273A mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 67 which comprises amino acids 18 to 732 of human ACE2 (SEQ ID No. 2) with a S645C mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 68 which comprises amino acids 18 to 740 of human ACE2 (SEQ ID No. 3) with a S645C mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 69 which comprises amino acids 18 to 732 of human ACE2 (SEQ ID No. 2) with a S645C mutation, a H374N mutation and a H378N mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 70 which comprises amino acids 18 to 740 of human ACE2 (SEQ ID No. 3) with a S645C mutation, a H374N mutation and a H378N mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 71 which comprises amino acids 18 to 732 of human ACE2 (SEQ ID No. 2) with a S645C mutation and a R273A mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 72 which comprises amino acids 18 to 740 of human ACE2 (SEQ ID No. 3) with a S645C mutation and a a R273A mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 73 which comprises amino acids 18 to 732 of human ACE2 (SEQ ID No. 2), the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 74 which comprises amino acids 18 to 740 of human ACE2 (SEQ ID No. 3), the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 75 which comprises amino acids 18 to 732 of human ACE2 (SEQ ID No. 2), the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 76 which comprises amino acids 18 to 740 of human ACE2 (SEQ ID No. 3), the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 77 which comprises amino acids 18 to 732 of human ACE2 (SEQ ID No. 2) with a H374N and H378N mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 78 which comprises amino acids 18 to 740 of human ACE2 (SEQ ID No. 3) with a H374N and H378N mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 79 which comprises amino acids 18 to 732 of human ACE2 (SEQ ID No. 2) with a H374N and H378N mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 80 which comprises amino acids 18 to 740 of human ACE2 (SEQ ID No. 3) with a H374N and H378N mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 81 which comprises amino acids 18 to 732 of human ACE2 (SEQ ID No. 2) with a R273A mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 82 which comprises amino acids 18 to 740 of human ACE2 (SEQ ID No. 3) with a R273A mutation, the numbering referring to SEQ ID No. 1, the linking sequence according to SEQ ID No. 15 and the variant of the Fc portion of human IgG1 according to SEQ ID No. 59.

In a particular embodiment, the fusion protein of the present invention has the amino acid sequence according to SEQ ID No. 83 which comprises amino acids 18 to 732 of human ACE2 (SEQ ID No. 2) with a R273A mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 84 which comprises amino acids 18 to 740 of human ACE2 (SEQ ID No. 3) with a R273A mutation, the numbering referring to SEQ ID No. 1, the linking sequence according to SEQ ID No. 4 and the variant of the Fc portion of human IgG4 according to SEQ ID No. 60.

In a particular embodiment, the fusion protein of the present invention has the amino acid sequence according to SEQ ID No. 85 which comprises amino acids 18 to 732 of human ACE2 (SEQ ID No. 2) with a R273A mutation, the numbering referring to SEQ ID No. 1, the linking sequence according to SEQ ID No. 15 and the Fc portion of human IgG1 according to SEQ ID No. 16.

In a particular embodiment, the fusion protein of the present invention has the amino acid sequence according to SEQ ID No. 86 which comprises amino acids 18 to 740 of human ACE2 (SEQ ID No. 3) with a R273A mutation, the numbering referring to SEQ ID No. 1, the linking sequence according to SEQ ID No. 15 and the Fc portion of human IgG1 according to SEQ ID No. 16.

In a particular embodiment, the fusion protein of the present invention has the amino acid sequence according to SEQ ID No. 87 which comprises amino acids 18 to 732 of human ACE2 (SEQ ID No. 2) with a R273A mutation, the numbering referring to SEQ ID No. 1, the linking sequence according to SEQ ID No. 4 and the Fc portion of human IgG4 according to SEQ ID No. 5.

In a particular embodiment, the fusion protein of the present invention has the amino acid sequence according to SEQ ID No. 88 which comprises amino acids 18 to 740 of human ACE2 (SEQ ID No. 3) with a R273A mutation, the numbering referring to SEQ ID No. 1, the linking sequence according to SEQ ID No. SEQ ID No. 4 and the Fc portion of human IgG4 according to SEQ ID No. 5.

In a particular embodiment, the fusion protein of the present invention has the amino acid sequence according to SEQ ID No. 89 which comprises amino acids 18 to 732 of human ACE2 (SEQ ID No. 2) with a R273A mutation, the numbering referring to SEQ ID No. 1, the linking sequence according to SEQ ID No. 4 and the variant of the Fc portion of human IgG4 according to SEQ ID No. 44.

In a particular embodiment, the fusion protein of the present invention has the amino acid sequence according to SEQ ID No. 90 which comprises amino acids 18 to 740 of human ACE2 (SEQ ID No. 3) with a R273A mutation, the numbering referring to SEQ ID No. 1, the linking sequence according to SEQ ID No. SEQ ID No. 4 and the variant of the Fc portion of human IgG4 according to SEQ ID No. 44.

In a particular embodiment, the fusion protein of the present invention has the amino acid sequence according to SEQ ID No. 93 which comprises amino acids 18 to 732 of human ACE2 (SEQ ID No. 2), the numbering referring to SEQ ID No. 1, the linking sequence according to SEQ ID No. 4 and the variant of the Fc portion of human IgG4 according to SEQ ID No. 44.

In a particular embodiment, the fusion protein of the present invention has the amino acid sequence according to SEQ ID No. 94 which comprises amino acids 18 to 740 of human ACE2 (SEQ ID No. 3), the numbering referring to SEQ ID No. 1, the linking sequence according to SEQ ID No. SEQ ID No. 4 and the variant of the Fc portion of human IgG4 according to SEQ ID No. 44.

The present invention also relates to a nucleic acid molecule comprising a nucleic acid sequence encoding the fusion protein of the present invention. The skilled person knows how to construct a nucleic acid molecule when the amino acid sequence of a protein is known. In particular, the construction of the nucleic acid molecule involves back-translating the amino acid sequence of the protein into a nucleic acid sequence using the three-letter genetic code and optionally taking into account the codon usage of the host cell in which the protein is to be expressed using the nucleic acid molecule.

In one embodiment, the nucleic acid molecule comprising a nucleic acid sequence encoding the fusion protein of the present invention is an isolated nucleic acid molecule. The term “isolated nucleic acid molecule” refers to a nucleic acid molecule that is identified and separated from at least one contaminant nucleic acid molecule with which it is ordinarily associated in the environment in which it was produced. Preferably, the isolated nucleic acid is free of association with all components associated with the production environment.

The term “vector,” as used herein, refers to a nucleic acid molecule capable of propagating another nucleic acid to which it is linked. The term includes the vector as a self- replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

Expression vectors contain elements for the expression of the nucleic acids such as suitable promoters and polyadenylation signals. In addition, the expression vectors typically contain a selection marker gene under the control of suitable promoter to enable the distinction of cells which contain the expression vector from cells which do not contain the expression vector. The elements and methods needed to construct expression vectors which are suitable for expressing a recombinant protein such as the fusion protein of the present invention are well-known to the skilled person and described for example in Makrides et al. (1999) Protein Expr. Purif. 17: 183-202 and Kaufman (2000) Mol. Biotechnol. 16: 151-161.

The expression vector is used to transform, i.e. genetically modify, suitable host cells. The skilled person is aware of methods for introducing the expression vectors into the mammalian cells. These methods include the use of commercially available transfection kits such as Lipofectamine® of ThermoFisher, PElmax of Polyplus Sciences), 293-Free transfection reagent (Millipore) or Freestyle Max of Invitrogen. Further suitable methods include electroporation, calcium phosphate-mediated transfection and DEAE-dextrane transfection. After transfection the cells are subjected to selection by treatment with a suitable agent based on the selection marker(s) encoded by the expression vector(s) to identify the stably transfected cells which contain the recombinant nucleic acid molecule.

The terms “host cell,” “host cell line,” and “host cell culture” are used interchangeably and refer to cells into which an exogenous nucleic acid or an expression vector has been introduced, including the progeny of such cells. Host cells include “transformants” and “transformed cells,” which include the primary transformed cell and progeny derived therefrom without regard to the number of passages.

The fusion protein of the present invention is preferably produced in mammalian host cells. Suitable mammalian host cells for expressing the fusion protein of the invention include Chinese Hamster Ovary (CHO) cells (including dhfr negative CHO cells used with a DHFR selectable marker), NS0 myeloma cells, COS cells, SP2 cells, monkey kidney CV1, human embryonic kidney line 293, baby hamster kidney cells (BHK), mouse Sertoli cells (TM4), African green monkey kidney cells (VERO-76), human cervical carcinoma cells (HELA), canine kidney cells (MDC), buffalo rat liver cells (BRL 3 A), human lung cells (W138), human liver cells (Hep G2), mouse mammary tumor cells (MMT 060562), TRI cells, MRC 5 cells and FS4 cells. More preferably, the host cells are derived from a rodent. Most preferably, the mammalian cells are Chinese hamster ovary (CHO) cells.

To produce the fusion protein of the present invention, the host cells are cultured in a suitable culture medium.

The terms “medium”, “cell culture medium” and “culture medium” are interchangeably used herein and refer to a solution containing nutrients which are required for growing mammalian cells. Typically, a cell culture medium provides essential and non-essential amino acids, vitamins, energy sources, lipids, and trace elements required by the cell for minimal growth and/or survival. The cell culture medium may also comprise growth factors. Preferably, the medium is chemically defined in that all its components and their concentration are known. Also preferably, the medium is serum-free and hydrolysate-free and does not contain any components derived from animals. In a more preferred embodiment the medium is serum-free and hydrolysate-free and does not contain any components derived from animals or insulin.

In one embodiment the medium used in the method of the present invention is a commercially available medium such as FreeStyle 293 expression medium (Life Technologies), PolCHO P Powder Base CD, ActiPro (both available from GE), PowerCHO-2, ProCHO-5 (both available from Lonza) or EX-CELL® Advanced CHO fed batch medium (available from Sigma).

For culturing the mammalian cells different strategies are available, including batch culture, perfusion culture, continuous culture and fed-batch culture. Preferably, a fed-batch culture process is used. In fed-batch culture the culturing process is started with a certain volume of the basal medium and one or more feed media comprising one or more nutrients are fed at later time-point(s) of the culture process to prevent nutrient depletion while no product is removed from the cell culture broth. Accordingly, the term “feeding” means that at least one component is added to an existing culture of cells.

The term “basal medium” is intended to refer to the medium which is used from the beginning of the cell culture process. The mammalian cells are inoculated into the basal medium and grown in this medium for a certain period until the feeding is started. The basal medium meets the definition of the culture medium as provided above. If a commercially available medium is used, additional components may be added to the basal medium.

The feed medium is added to the cell culture after the cells have been cultured in the basal medium for a certain period. The feed medium serves to prevent nutrient depletion and therefore may not have the same composition as the basal medium. In particular, the concentration of one or more nutrients may be higher in the feed medium than in the basal medium. In one embodiment, the feed medium has the same composition as the basal medium. In another embodiment, the feed medium has another composition as the basal medium. The feed medium may be added continuously or as a bolus at defined time points.

Suitable feed media are known to the skilled person and include PolCHO Feed-A Powder Base CD, PolCHO Feed-B Powder Base CD, Cell Boost 7a and Cell Boost 7b (all available from GE), BalanCD® CHO Feed 3 Medium (available from Irvine Scientific) and EX-CELL® Advanced CHO feed 1 (available from Sigma).

The culturing of the host cell may be performed at a constant temperature, e.g. at a temperature of 37° C.±0.2° C. Alternatively, the culture temperature may be reduced from a first temperature to a second temperature, i.e. the temperature is actively downregulated. Hence, the second temperature is lower than the first temperature. The first temperature may be 37° C.±0.2° C. The second temperature may be in the range of from 30° C. to 36° C.

After the fusion protein of the present invention has been produced by culturing the host cell in a suitable culture medium, the fusion protein is harvested from the cell culture. Since Fc fusion proteins expressed from mammalian cells are typically secreted into the cell culture fluid during the cultivation process, the product harvest at the end of the cultivation process occurs by separating cell culture fluid comprising the fusion protein from the cells. The cell separation method should be gentle to minimize cell disruption to avoid the increase of cell debris and release of proteases and other molecules that could affect the quality of the fusion protein product. Usually, the harvesting of the cell culture fluid comprising the fusion protein involves centrifugation and/or filtration, whereby the fusion protein is present in the supernatant and the filtrate, respectively. Expanded bed adsorption chromatography is an alternative method to avoid centrifugation/filtration methods.

After harvesting the cell culture fluid comprising the fusion protein the fusion protein has to be purified from the cell culture fluid. The purification of Fc fusion proteins is usually accomplished by a series of standard techniques that can include chromatographic steps such as anion exchange chromatography, cation exchange chromatography, affinity chromatography and in particular protein A affinity chromatography, hydrophobic interaction chromatography, hydroxyapatite chromatography and size exclusion chromatography. Further, the purification process may comprise one or more ultra-, nano- or diafiltration as well as tangential flow filtration and/or cross flow filtration steps.

After purifying the fusion protein it can be used to prepare a pharmaceutical composition. A pharmaceutical composition is a composition which is intended to be delivered to a patient for treating or preventing a disease or condition. In addition to the active agent, i.e. the fusion protein of the present invention, a pharmaceutical composition typically contains at least one pharmaceutically acceptable excipient. Pharmaceutically acceptable excipients are substances which do not interfere with the physiological activity of the fusion protein and which stabilize the pharmaceutical composition and/or enhance solubility or decrease viscosity of the pharmaceutical composition. Typical pharmaceutically acceptable excipients for recombinant proteins include buffers, salts, sugars or sugar alcohols, amino acids and surface-active agents.

The pharmaceutical composition comprises a therapeutically effective amount of the fusion protein of the present invention. The term “therapeutically effective amount” refers to an amount of the fusion protein of the present invention sufficient to treat a specified disorder, condition or disease such as ameliorate, palliate, lessen, and/or delay one or more of its symptoms. With respect to an infection with a coronavirus and in particular SARS-CoV-2 the therapeutically effective amount of the fusion protein of the present invention ameliorates, palliates, lessens, and/or delays one or more of symptoms selected from coughing, shortness of breath, difficulty breathing, fever, chills, tiredness, muscle aches, sore throat, headache, chest pain and loss of smell and/or taste. A therapeutically effective amount can be administered in one or more administrations.

The fusion protein of the present invention is for medical use, i.e. it is intended to be used to prevent and/or treat a disease.

As used herein,“treatment” or“treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, and/or prolonging survival. The use of the present invention contemplates any one or more of these aspects of treatment.

The term “prevent,” and similar words such as “prevented”, “preventing” etc., indicate an approach for preventing, inhibiting, or reducing the likelihood of the recurrence of, a disease or condition. It also refers to delaying the recurrence of a disease or condition or delaying the recurrence of the symptoms of a disease or condition. As used herein, “prevention” and similar terms also includes reducing the intensity, effect, symptoms and/or burden of a disease or condition prior to recurrence of the disease or condition.

In one embodiment, the fusion protein of the present invention is used to prevent and/or treat an infection with a coronavirus binding to ACE2. Coronaviruses are enveloped viruses with a positive sense, single-stranded RNA genome and an icosahedral protein shell. The Spike protein consisting of the S1 and S2 subunits forms a homotrimer which projects from the envelope and mediates the interaction with the target cells by binding to ACE2. Coronaviruses often cause respiratory diseases in humans and other mammalian as well as bird species. In humans, seven coronavirus strains are known: HCoV-OC43, HCoV-HKU1, HCoV-229E, HCoV-NL63, MERS-CoV, SARS-CoV and SARS-CoV-2. The first four coronavirus strains (HCoV-OC43, HCoV-HKU1, HCoV-229E, HCoV-NL63) cause only mild symptoms, whereas infection with MERS-CoV, SARS-CoV and SARS-CoV-2 may lead to severe, potentially life-threatening disease.

It has been shown that SARS-CoV, SARS-CoV-2 and HCoV-NL63 bind to ACE2 and use this binding to enter the target cells (Li et al. (2003) Nature 426(6965): 450-4; Hoffmann et al. (2020) Cell 181: 1-10; Hofmann et al. (2005) Proc Natl Acad Sci USA. 102(22):7988-93). Accordingly, the fusion protein of the present invention can be used to treat and/or prevent infection with a coronavirus binding to ACE2, in particular infection with SARS-CoV, SARS-CoV-2 or HCoV-NL63. Further coronaviruses binding to ACE2 can be identified by inoculating cells expressing ACE2 either transiently or constitutively with pseudotyped VSV (vesicular stomatitis virus) expressing the coronavirus Spike protein and a reporter protein and detecting the activity of the reporter protein after the inoculation period (see protocol in Hoffmann et al. (2020) Cell 181: 1-10). In one embodiment, the fusion protein of the present invention is used to treat and/or prevent infection with a coronavirus binding to ACE2, wherein the coronavirus binding to ACE2 is not SARS-CoV.

In one embodiment, the fusion protein of the present invention is used to treat and/or prevent infection with a coronavirus binding to ACE2, wherein the coronavirus binding to ACE2 is SARS-CoV-2 or a variant of SARS-CoV-2 comprising the amino acid substitution D614G and/or the amino acid substitution N439K. The variant of SARS-CoV-2 comprising the amino acid substitution D614G is described in Korber et al. (2020) Cell 182(4): 812-827 and the amino acid substitution N439K is described in Thomson et al. (2021) Cell 184(5): 1171,1187.e20; available at https://doi.org/10.1101/2020.11.04.355842. In one embodiment, the fusion protein of the present invention is used to treat and/or prevent infection with a coronavirus binding to ACE2, wherein the coronavirus binding to ACE2 is a variant of SARS-CoV-2 comprising the amino acid substitution D614G. The amino acid substitution D614G is caused by an A-to-G nucleotide mutation at position 23,403 in the Wuhan reference strain. The numbering of the amino acids in the variants refers to the numbering in the Spike protein of SARS-CoV-2 according to SEQ ID No. 18. Hence, a SARS-CoV-2 virus with the Spike protein according to SEQ ID No. 18 is defined to be the wild-type SARS-CoV-2 from which any variants are derived.

In one embodiment, the fusion protein of the present invention is used to treat and/or prevent infection with a coronavirus binding to ACE2, wherein the coronavirus binding to ACE2 is a variant of SARS-CoV-2 comprising the amino acid substitution D614G and at least one additional amino acid substitution. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, N501Y, A570D, P681H, T7161, S982A and D1118H and comprising a deletion of amino acids 69, 70 and 145. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, Y453F, 1692V and M1229I and comprising a deletion of amino acids 69 and 70. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, S131, W152C and L452R. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, E484K and V1176F. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, L18F, T20N, P26S, D138Y, R190S, K417T, E484K, N501Y, H655Y, T1027I and V1176F. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, D80A, D215G, K417N, E484K, N501Y and A701V and comprising a deletion of amino acids 242, 243 and 244. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, L18F, D80A, D215G, K417N, E484K, N501Y and A701V and comprising a deletion of amino acids 242, 243 and 244. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions D614G, D80A, R246I, K417N, E484K, N501Y and A701V and comprising a deletion of amino acids 242, 243 and 244. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions E484Q and L452R. In one embodiment, the fusion protein of the present invention is used to treat and/or prevent infection with a coronavirus binding to ACE2, wherein the coronavirus binding to ACE2 is a variant of SARS-CoV-2 comprising the amino acid substitutions E484K and D614G and comprising a deletion of amino acids 145 and 146.

The numbering of the amino acids in the variants refers to the numbering in the Spike protein of SARS-CoV-2 according to SEQ ID No. 18. For the purposes of defining variants, the amino acid sequence according to SEQ ID NO. 18 is considered to be the wild-type sequence of the Spike protein of SARS-CoV-2.

In one embodiment, the fusion protein of the present invention is used to treat and/or prevent infection with a coronavirus binding to ACE2, wherein the coronavirus binding to ACE2 is a variant of SARS-CoV-2 comprising one or more amino acid substitutions in the receptor-binding domain of the Spike protein of SARS-CoV-2. The receptor-binding domain of the Spike protein of SARS-CoV-2 comprises amino acids 331 to 524 of SEQ ID No. 18 (see Tai et al. (2020) Cell. Mol. Immunol. 17: 613-620). In one embodiment, the fusion protein of the present invention is used to treat and/or prevent infection with a coronavirus binding to ACE2, wherein the coronavirus binding to ACE2 is a variant of SARS-CoV-2 comprising the amino acid substitution N501Y. In one embodiment, the fusion protein of the present invention is used to treat and/or prevent infection with a coronavirus binding to ACE2, wherein the coronavirus binding to ACE2 is a variant of SARS-CoV-2 comprising the amino acid substitution E484K. In one embodiment, the fusion protein of the present invention is used to treat and/or prevent infection with a coronavirus binding to ACE2, wherein the coronavirus binding to ACE2 is a variant of SARS-CoV-2 comprising the amino acid substitution K417T/N.

In one embodiment, the fusion protein of the present invention is used to treat and/or prevent infection with a coronavirus binding to ACE2, wherein the coronavirus binding to ACE2 is a variant of SARS-CoV-2 which has a higher binding affinity to ACE2 compared to the SARS-CoV-2 comprising the wild-type Spike protein according to SEQ ID No. 18. The affinity of a variant of SARS-CoV-2 to ACE2 can for example be determined using a pseudovirus assay. The pseudovirus assay uses a lentivirus which is pseudotyped with the S protein of wild-type SARS-CoV-2 or of a variant thereof and which contains a reporter gene such as the luciferase gene. Such lentiviruses can be obtained for example from BPS Bioscience. The pseudotyped lentivirus is incubated with ACE2 expressing cells to allow the virus to enter the cells and to express the reporter gene. If the expression of the reporter gene from a lentivirus pseudotyped with the S protein of a variant SARS-CoV-2 is higher than the expression of the reporter gene from a lentivirus pseudotyped with the S protein of wild-type SARS-CoV-2, the variant has a higher binding affinity to ACE2. In the context of the present invention it has been found that the fusion proteins of the present invention have a higher affinity to those variants which have a higher binding affinity to ACE2, such as the variant B.1.1.7.

In one embodiment, the fusion protein of the present invention is used to treat and/or prevent infection with a coronavirus binding to ACE2, wherein the coronavirus binding to ACE2 is a variant of SARS-CoV-2 which has a higher transmissibility compared to the SARS-CoV-2 comprising the wild-type Spike protein according to SEQ ID No. 18. The viral transmissibility can be determined using the basic reproduction number R₀ which is the average number of people who will catch a disease from one contagious person.

The route of administration is in accordance with known and accepted methods, e.g., injection or infusion by subcutaneous, intravenous, intraperitoneal, intramuscular, intra-arterial, intralesional or intraarticular routes. In another embodiment the fusion protein of the present invention is to be administered intranasally, e.g. by means of a nasal spray, a nasal ointment or nasal drops. In another embodiment, the fusion protein of the present invention is administered by topical administration or by inhalation. Preferably, the fusion protein of the present invention is administered by intravenous injection or infusion.

Dosages and desired drug concentrations of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp.42-46.

In one embodiment, the fusion protein of the present invention is administered intravenously at a dosage of 0.1 mg/kg body weight to 4 mg/kg body weight, such as a dosage of 0.1 mg/kg body weight, 0.2 mg/kg body weight, 0.3 mg/kg body weight, 0.4 mg/kg body weight, 0.5 mg/kg body weight, 0.6 mg/kg body weight, 0.7 mg/kg body weight, 0.8 mg/kg body weight, 0.9 mg/kg body weight, 1.0 mg/kg body weight, 1.1 mg/kg body weight, 1.2 mg/kg body weight, 1.3 mg/kg body weight, 1.4 mg/kg body weight, 1.5 mg/kg body weight, 1.6 mg/kg body weight, 1.7 mg/kg body weight, 1.8 mg/kg body weight, 1.9 mg/kg body weight, 2.0 mg/kg body weight, 2.1 mg/kg body weight, 2.2 mg/kg body weight, 2.3 mg/kg body weight, 2.4 mg/kg body weight, 2.5 mg/kg body weight, 2.6 mg/kg body weight, 2.7 mg/kg body weight, 2.8 mg/kg body weight, 2.9 mg/kg body weight, 3.0 mg/kg body weight, 3.1 mg/kg body weight, 3.2 mg/kg body weight, 3.3 mg/kg body weight, 3.4 mg/kg body weight, 3.5 mg/kg body weight, 3.6 mg/kg body weight, 3.7 mg/kg body weight, 3.8 mg/kg body weight, 3.9 mg/kg body weight or 4.0 mg/kg body weight.

In one embodiment, the fusion protein of the present invention is administered intravenously at a dosage of 10 mg/kg body weight to 150 mg/kg body weight, such as a dosage of 10 mg/kg body weight, 15 mg/kg body weight, 20 mg/kg body weight, 25 mg/kg body weight, 30 mg/kg body weight, 35 mg/kg body weight, 40 mg/kg body weight, 45 mg/kg body weight, 50 mg/kg body weight, 55 mg/kg body weight, 60 mg/kg body weight, 65 mg/kg body weight, 70 mg/kg body weight, 75 mg/kg body weight, 80 mg/kg body weight, 85 mg/kg body weight, 90 mg/kg body weight, 95 mg/kg body weight, 100 mg/kg body weight, 105 mg/kg body weight, 110 mg/kg body weight, 115 mg/kg body weight, 120 mg/kg body weight, 125 mg/kg body weight, 130 mg/kg body weight, 135 mg/kg body weight, 140 mg/kg body weight, 145 mg/kg body weight or 150 mg/kg body weight.

The fusion protein may be administered once per day, twice per day, three times per day, every other day, once per week or once every two weeks.

The fusion protein may be administered for a period of three days, four days, five days, six days, seven days, eight days, nine days or ten days.

By administering the fusion protein of the present invention, the infection with a coronavirus and in particular with SARS-CoV-2 is treated, i.e. at least one of the symptoms of an infection with SARS-CoV-2 is reduced or abolished. Symptoms of an infection with SARS-CoV-2 include coughing, shortness of breath, difficulty breathing, fever, chills, tiredness, muscle aches, sore throat, headache, chest pain and loss of smell and/or taste. In one embodiment, by the administration of the fusion protein of the present invention the fever caused by infection with SARS-CoV-2 is reduced. In one embodiment, the administration of the fusion protein of the present invention to a subject reduces the risk that the subject experiences a severe course of infection with SARS-CoV-2. In one embodiment, the administration of the fusion protein of the present invention to a subject reduces the risk that the subject experiences multi-organ failure, acute respiratory distress syndrome (ARDS) or pneumonia. In one embodiment, the administration of the fusion protein of the present invention to a subject reduces the risk that the subject experiences long-term effects of the infection with SARS-CoV-2 such as lung damage, neurological disorders, dermatological disorders and cardiovascular disease. In one embodiment, the administration of the fusion protein of the present invention to a subject reduces the concentration of the cytokines IL6 and/or IL8 in the blood. In one embodiment, the administration of the fusion protein of the present invention to a subject reduces the concentration of SARS-CoV-2 virus particles in the blood. In one embodiment, the administration of the fusion protein of the present invention to a subject stimulates the production of antiviral antibodies. In one embodiment, the administration of the fusion protein of the present invention to a subject stimulates the production of antiviral IgA and/or IgG antibodies.

In one embodiment, the fusion protein of the present invention is administered to a subject suffering from a severe infection with SARS-CoV-2. In one embodiment, the fusion protein of the present invention is administered to a subject infected with SARS-CoV-2 and requiring artificial ventilation. In one embodiment, the fusion protein of the present invention is administered to a subject infected with SARS-CoV-2 and requiring extracorporeal membrane oxygenation (ECMO).

By administering the fusion protein of the present invention, the infection with a coronavirus and in particular with SARS-CoV-2 is prevented, i.e. the treated subject does not develop symptoms of an infection with SARS-CoV-2.

In one embodiment, the fusion protein of the present invention is administered to a subject which has been in contact with a subject infected with SARS-CoV-2. Subjects which have been in contact with a subject infected with SARS-CoV-2 can be identified by use of a “Corona warning app” installed on the smartphone.

In one embodiment, the fusion protein of the present invention is administered to a subject for which a test with a throat or nasal swab of said subject indicates that it is infected with SARS-CoV-2, but which has not developed any symptoms of an infection with SARS-CoV-2.

In the treatment or prevention of an infection with a coronavirus binding to ACE2 and in particular SARS-CoV-2 the fusion protein of the present invention may be combined with a known anti-viral agent. Anti-viral agents are medicaments used to treat viral infections and include both specific anti-viral agents and broad-spectrum viral agents. Suitable anti-viral agents include, but are not limited to, nucleoside analoga, inhibitors of viral protease, inhibitors of viral polymerase, blockers of virus entry into the cell, Janus kinase inhibitors, but also inhibitors of inflammatory mediators.

In specific embodiments, the anti-viral agent is selected from the group consisting of remdesivir, arbidol HCl, ritonavir, lopinavir, darunavir, ribavirin, chloroquin and derivatives thereof such as hydroxychloroquin, nitazoxanide, camostat mesilate, anti-IL6 and anti-IL6 receptor antibodies such as tocilizumab, siltuximab and sarilumab and baricitinib phosphate.

In the treatment or prevention of an infection with SARS-CoV-2 the fusion protein of the present invention may be combined with an anti-SARS-CoV-2 monoclonal antibody. Anti-SARS-CoV2 monoclonal antibodies include, but are not limited to, bamlanivimab (LY-CoV555 20; LY3819253;) developed by Eli Lilly and Company, etesevimab (LY-3832479; LY-COV016; JS-016; NP-005), REGN-COV2 which is a cocktail of REGN10933 (casivirimab) and REGN10987 (imdevimab) and which is developed by Regeneron, sotrovimab (VIR-7831; GSK4182136;) developed by Vir Biotechnology and GlaxoSmithKline, CT-P59 developed by Celltrion, AZD 7442 which is a combination of antibodies AZD8895 and AZD1061 and which is developed by Astra Zeneca, JS016 developed by Junshi Biosciences, TY027 developed 25 by Tychan Pte Ltd, BRII-96 and BRII-98 developed by Brii Biosciences, SCTA01 developed by Sinocelltech Ltd, ADM03820 developed by Ology Bioservices, BI767551 developed by Boehringer Ingelheim and others and COR-101 developed by Corat Therapeutics.

Apart from its function in binding coronaviruses, ACE2 has also been implicated in several disorders and diseases such as hypertension (including high blood pressure), congestive heart failure, chronic heart failure, acute heart failure, contractile heart failure, myocardial infarction, arteriosclerosis, kidney failure, renal failure, Acute Respiratory Distress Syndrome (ARDS), Acute Lung Injury (ALI), chronic obstructive pulmonary disease (COPD), pulmonary hypertension, renal fibrosis, chronic renal failure, acute renal failure, acute kidney injury, inflammatory bowel disease and multi-organ dysfunction syndrome. Hence, the fusion protein of the present invention can also be used in the treatment of these disorders and diseases.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

The detailed description is merely exemplary in nature and is not intended to limit application and uses. The following examples further illustrate the present invention without, however, limiting the scope of the invention thereto. Various changes and modifications can be made by those skilled in the art on the basis of the description of the invention, and such changes and modifications are also included in the present invention.

### EXAMPLES

### A. Materials and Methods

### 1. Construction of Fusion Proteins

Four fusion proteins of the present invention were constructed. In addition, four fusion proteins comprising the Fc portion of human IgG1 instead of the Fc portion of human IgG4 were constructed as comparative examples. The following Table 1 shows the parts of the fusion proteins.

TABLE 1
Parts of the fusion proteins tested

| Fusion | | Enzymatically | | Fc | SEQ ID |
|---|---|---|---|---|---|
| protein no. | ACE2 | active | Linker | portion | No. |
| 1 | 18-732 | + | IgG4 | IgG4 | 6 |
| 2 | 18-740 | + | IgG4 | IgG4 | 7 |
| 3 | 18-732 | − | IgG4 | IgG4 | 8 |
| 4 | 18-740 | − | IgG4 | IgG4 | 9 |
| 5 | 18-732 | + | IgG1 | IgG1 | 10 |
| 6 | 18-740 | + | IgG1 | IgG1 | 11 |
| 7 | 18-732 | − | IgG1 | IgG1 | 12 |
| 8 | 18-740 | − | IgG1 | IgG1 | 13 |

The nucleic acid sequence encoding the construct was inserted into a variant of the expression vector pcDNA3.1 (Invitrogen V860-20) using Hindlll/Xhol restriction enzymes. The albumin signal sequence according to SEQ ID No. 17 was attached to the N-terminus of the constructs. The expression vector was then used to transiently transfect 293 cells using the FreeStyle expression system (available from ThermoFisher). On day six samples were analyzed for cell viability and cell density and supernatants were harvested by two step centrifugation and were sterile-filtered. The material was pooled and half of it was stored at −80° C. until purification. The other half was subjected to Protein A purification. Additionally, small samples (˜0.5 mL) were taken from the pools to determine expression by bio-layer interferometry (BLI).

### 2. Protein Purification

Purification of the transient material was performed by protein A column chromatography followed by preparative SEC. For protein A purification, after loading the sample, the column was washed and the ACE2-Fc fusion proteins were eluted using 40 mM NaAc, pH=3.0. Following elution, samples were first neutralized to pH=7.5 using 1M Tris, pH=9.0, subsequently diluted 1:1 with 50 mM Tris, pH=7.5, 300 mM NaCl and concentrated to 10 mg/mL using spin filters. Concentrated proteins were further purified with a Superdex 200 increase (GE Healthcare) column equilibrated with 50 mM Tris, pH=7.5, 150 NaCL. The main peak was pooled, the protein concentration was adjusted to 1 mg/mL, passed over a sterilizing filter and stored at 4° C. until further usage.

### 3. Determination of Protein Concentration (A280) by Slope Spectrometry

Purified material of the different ACE2-Fc fusion proteins was analyzed by slope spectroscopy to determine the protein content. Verification of absence of buffer interference was checked and by using a variable pathlength, the protein concentration was accurately measured without any prerequisites for sample predilution during the purification process.

### 4. Determination of High Molecular Weight Species by Analytical Size Exclusion Chromatography

The different purified protein constructs were analyzed by analytical Size Exclusion Chromatography (SEC). Briefly, samples were analyzed on a Waters H-Class bio UPLC system using an Acquity UPLC Protein BEH SEC column, 4.6 mm×150 mm, 200 Å, 1.7 μm. Detection was based on UV absorbance at 280 nm. 20 μg of sample was loaded, the mobile phase consisted of 20 mM sodium phosphate pH=7.0, 150 mM NaCl and proteins were eluted isocratically at a flow rate of 0.3 mL/min.

### 5. Stability Study

For all eight purified Fc-fusion proteins the stability study was performed using 740 μL (300 μL backup) aliquots at a concentration of 1 mg/mL in tightly closed vials. One vial of each Fc-fusion protein was incubated first for 3 weeks at 37° C. Subsequently, a second vial per Fc-fusion protein was added to the incubation. Moreover, after 2 additional weeks a 3rd vial was added to the incubation per Fc-fusion protein. The incubation lasted for one additional week so that the 1st set of vials were incubated for 6 weeks, the 2nd set of vials for 3 weeks and the 3rd set of vials for 1 week. Finally, in parallel to the last week of the stability study a 4th set of vials underwent 3×freeze-thaw (F/T) cycles at −80° C. All the samples from the test intervals, i.e. 6, 3 and 1 week together with the F/T samples were analysed using the method explained above for analytical SEC. For T=0, data from testing after purification was used.

### 6. Determination of O-Glycosylation by Pepmap

Purified ACE2-Fc fusion proteins were analyzed by peptide mapping using UPLC-RP/MS. Proteins were denatured in guanidine hydrochloride, followed by reduction with DTT for 1 hour at 4° C. and alkylation with iodoacetamide for 30 minutes at room temperature in the dark. Samples were subjected to a proteolytic digest with a cocktail of trypsin and Lys-C enzymes for 4 hours. Proteolytic peptides were separated on a C18 reversed phase UPLC (e.g. Peptide BEH C18, 2.1×300 mm, 300 Å, 1.7 μm) column using 0.1% formic acid in Milli-Q and acetonitrile as the mobile phases in a peptide mapping gradient of 60 minutes with an initial hold time of 4.5 minutes. Two different collision energies were applied to improve the coverage of glycopeptides (low collision energy from 15-30 eV and high collision energy from 60-100 eV). Detection of the peptides was performed by UV at 214 nm and by mass spectrometry using a Xevo G2-XS QToF mass spectrometer from Waters. Analysis was performed in MS^{E} mode to obtain peptide verification by fragmentation (MS/MS) in addition to mass verification by MS. MS^{E} spectra were processed using Waters UNIFI 1.9 software, which includes Waters MaxEnt3 for deconvolution. Glu1-Fibrinopeptide B was infused during the run using a separate reference probe, and used for lockmass correction. Deconvoluted masses were matched to the theoretical sequence with a 10 ppm ion tolerance for precursor ion masses, and 20 ppm for fragment ion masses. For the identification of glycosylated peptides, a limited library of C-, N- and O-glycans was included in the search. To increase confidence of the assignments, fragment spectra of glycosylated peptides were checked for the presence of marker ions (e.g. at m/z 292 and 204). In addition, for the sequence coverage maps, peptides with less than 3 fragment ions were excluded.

### 7. Determination of Binding of the Fusion Proteins to the Spike Protein of SARS-CoV-2

### a) Surface Plasmon Resonance with the Spike Protein of SARS-CoV-2 (Wild-Type)

The binding of the fusion proteins to the Spike protein of SARS-CoV-2 was analyzed by surface plasmon resonance (Biacore) using commercially available SARS-CoV-2 Spike protein (ACROBiosystems, Newark, USA).

### Materials

The SARS-CoV-2 RBD with his tag and an AviTag was from Acrobiosystems (Cat. No. SPD-C82E9, Lot. No. BV3541b-2043F1-RD). The protein was reconstituted according to the manufacturer's recommendations, aliquoted, shock frozen in liquid nitrogen and stored at −80° C. until use. A fresh aliquot was used for each measurement.

The running buffer was 1×HBS-EP+ prepared from 10×HBS-EP+ (Cytiva) by a 1:10 dilution with MilliQ water. The diluted buffer was filtered through a 0.1 μm filter.

The Biotin CAPture kit (Cytiva) and a Biacore X-100 system were used for the measurements.

### Methods

The ACE2-Fcs were diluted to 200 nM with 1×HBS-EP+ buffer. The concentration was verified by UV spectrometry in a 10 mm quartz cuvette, using an A_{280, 0.1%} of 1.84. The 200 nM ACE2-Fc was diluted with 1×HBS-EP+ to 40, 8, 1.6 and 0.32 nM. The thawed SARS-CoV-2 RBD domain was diluted 1:100 with 1×HBS-EP+ to a concentration of 2 μg/mL. A single cycle kinetic method was used. The flow was 30 μL/min. Before each measurement, three injections of regeneration solution were used to condition the chip. The immobilization time for the SARS-CoV-2 with AviTag (Acrobiosystems) was 30 s. After ligand immobilization, the different concentrations of ACE2-Fc (0.32, 1.6, 8, 40 and 200 nM) were injected over the immobilized ligand in a single-cycle kinetic mode, starting from low to high. The baseline was obtained from two cycles with buffer injections only.

The data was evaluated in the Biacore software with a 1:1 binding model that yields a K_{D}.

### b) Surface Plasmon Resonance with the Spike Protein of the Delta Variant of SARS-CoV-2

The ACE2-Fc constructs were diluted to 1 nM with 1×HBS-EP+ buffer. The thawed SARS COV-2 Spike Trimer variants (obtained from AcroBiosystems, catalogue number SPN-C82E7) were prediluted to 200 nM with 1×HBS-EP+ buffer and subsequently diluted with 1×HBS-EP+ buffer to the final assay concentrations of 20.00, 6.67, 2.22, 0.74 and 0.25 nM. Before the experiment the chip was immobilised with Anti-human IgG Fc antibody to a capture level of 1000 RU via a Amine coupling kit (NHS/EDC). Before each experiment 6 startup cycles with the ligand ACE2-FC construct as well as the analyte Spike Trimers were run in order to condition the chip. Before each sample injection a blank buffer sample was injected for subsequent blank subtraction. A single cycle method was used for the analysis. The flow rate was set to 20 μL/min and 1×HBS-EP+ was used as the running buffer. The immobilisation time for the ACE2-Fc constructs was 80 s, while the sample contact times were 600 s for the association and 900 s for the dissociation period. The Spike Trimer samples were injected over the immobilized ACE2-Fc constructs in a single-cycle kinetic assay, starting from low to high sample concentration in triplicate measurements. After each sample cycle the chip surface was regenerated with a 3M solution of MgCl₂. The kinetic values were obtained by a double referenced (Reference flow cell and blank buffer referenced) kinetic screen of the sensorgrams via the Biacore T200 Evaluation software. Baseline plots and capture level plots of each sample cycle were generated for assay verification.

### c) ELISA 1

To quantify the binding of the fusion proteins to the Spike protein of SARS-CoV-2 an ELISA assay was performed. The ELISA plate was coated with 0.2 μg/well of commercially available SARS-CoV-2 Spike protein (ACROBiosystems, Newark, USA) in coating buffer (15 mM Na₂CO₃, 35 mM NAHCO₃, 7.7 mM NaN₃, pH 9.6). The wells were washed four times with 300 μl per well washing buffer (0.05% Tween-20 in TBS, pH 7.4). The wells were blocked with 300 μl blocking buffer (2% BSA in washing buffer, pH 7.4) at 37° C. for 90 minutes, before the wells were washed four times with 300 μl per well washing buffer (0.05% Tween-20 in TBS, pH 7.4). Then 100 μl of serially diluted fusion protein were added to each well and the wells were incubated at 37° C. for one hour. The fusion protein was diluted in sample dilution buffer (0.5% BSA in washing buffer, pH 7.4). After incubation the wells were washed four times with 300 μl per well washing buffer (0.05% Tween-20 in TBS, pH 7.4).

Then, 100 μl of a horseradish peroxidase-conjugated anti-human Fc antibody (diluted in 0.5% BSA in washing buffer, pH 7.4) was added to each well and the wells were incubated at 37° C. for one hour. After incubation the wells were washed four times with 300 μl per well washing buffer (0.05% Tween-20 in TBS, pH 7.4), before 200 μl of horseradish peroxidase substrate (8 μl H₂O₂ and 100 μl 10 mg/ml TMB in 10 ml substrate solution A (50 mM Na₂HPO₄×12H₂O, 25 mM citric acid, pH 5.5) were added and the wells were incubated at 37° C. for twenty minutes in the dark. The reaction was terminated by adding 50 μl of 1 M sulfuric acid to each well. The plates were read in an ELISA reader at OD450 nm.

Inhibition of binding of SARS-CoV-2 Spike S1 protein to ACE2 was tested using the ACE2:SARS-CoV-2 Spike S1 Inhibitor Screening Assay Kit (BPS Bioscience; Catalog #79945) according to the instructions of the manufacturer with an adapted neutralization procedure. Briefly, biotinylated SARS-CoV-2 Spike S1 protein (25 nM) was incubated with serial dilutions of the ACE2-Fc fusion proteins in a 96-well neutralization plate at room temperature for one hour with slow shaking (=neutralization mix).

ACE2 protein was attached to a nickel-coated 96-well plate at a concentration of 1 μg/mL and incubated at room temperature for one hour with slow shaking. Unbound ACE2 was removed by a washing step. Subsequently, the neutralization mix was transferred to the ACE2 coated plate and the plate was incubated at room temperature for one hour with slow shaking. Following a 10 min blocking step, the plate was incubated for 1 hour at room temperature with slow shaking with Streptavidin-HRP. Following a washing and a 10 min blocking, HRP substrate was added and the plate was analyzed on a chemiluminescence reader.

### d) ELISA 2

To quantify the binding of the fusion protein to the Spike protein of SARS-CoV-2 an ELISA assay was performed. The ELISA plate (NUNC) was coated with 1.0 μg/mL of commercially available SARS-CoV-2 Spike protein (SPN-C52H9, ACROBiosystems) using 100 μL/well in coating buffer (PBS). The wells were incubated over night at 4° C. Next day, the coating was removed and the wells were washed three times with 300 μl per well of washing buffer (10 mM sodium phosphate, 150 mM NaCl, 0.05% Tween-20, pH=7.5). The wells were blocked with 200 μl blocking buffer (wash buffer supplemented with 1% BSA) at room temperature for one hour, while shaking at 150 rpm. Afterwards the blocking buffer was removed and 100 μl of serially diluted fusion protein were added to each well and the wells were incubated at room temperature for one hour at 150 rpm. The fusion protein was diluted in sample dilution buffer (1% BSA in washing buffer). After incubation the wells were washed three times with 300 μl per well washing buffer. Next, 100 μl of a horseradish peroxidase-conjugated anti-human IgG4Fc antibody (Southern Biotech, 9200-05, diluted 1:4000 in blocking buffer) was added to each well and the wells were incubated at room temperature for one hour while shaking. After incubation, the wells were washed three times with 300 μl per well of washing buffer, before 100 μl of TMB solution (Invitrogen, SB02) was added and the wells were incubated at room temperature for two minutes. The reaction was stopped with 100 μl/well 1 M HCl and incubated for 30 seconds at room temperature protected from light while shaking. After further incubation for 15 minutes at room temperature in the dark, the plates were read in a microplate reader (Synergy HTX, BioTek) at OD450 nm with a reference at OD655. The concentrations (in μg/mL) were plotted against OD450 (after background subtraction) using a 4-parameter logistic curve fit model.

### 8. Analysis of Infection with SARS-CoV-2 Strain Victoria/1/2020 in the Presence of the Fusion Proteins

The analysis of infection with SARS-CoV-2 was performed with Vero cells in the absence or presence of the fusion proteins. Infection with SARS-CoV-2 was detected by determining the number of immunoplaques.

VeroE6 cells were plated in a 96-well plate and incubated overnight. Six two-fold serial dilutions of the ACE2-Fc fusion protein samples were prepared in 96-well transfer plate(s). The Victoria/1/2020 SARS-CoV-2 wild-type virus was added sequentially to the dilutions at a target working concentration of approximately 100 plaque-forming units [PFU]/well and incubated at 37° C. for 60 to 90 minutes. Following the incubation period, the neutralization mixture was transferred to the assay plates with VeroE6 cells and subsequently incubated at 37° C. and 5% CO2. Following a 60 to 90 minute incubation period, carboxymethyl cellulose (CMC) overlay medium was added to the wells and the plates were incubated for another 24 hours. Then, the cells were fixed and stained using an antibody pair specific for the SARS-CoV-2 RBD S protein. Immunoplaques were visualized using TrueBlueTM substrate and counted using an Immunospot Analyzer (CTL). The immunoplaque counts were exported to SoftMax Pro (Molecular Devices) and the neutralizing titer of a serum sample was calculated as the reciprocal dilution corresponding to the 50% neutralization titer (ID50) for that specific sample.

### 9. ACE2 Activity Assay

An ACE2 activity assay kit from Abcam (ab273297) was used to measure the enzymatic activity of the constructs. The assay was performed according to manufacturer's manual. Two commercially available ACE2-Fc fusion proteins (from Genscript (Cat.No. Z03484-1) and Acrobiosystems (Cat.No. AC2-H5257) were used as reference proteins (Ref1 and Ref2). The assay is based on the cleavage of a synthetic peptidyl-MCA derivate. This substrate is cleaved by the active ACE2 to release the free MCA fluorophore that has an increased fluorescence intensity at 420 nm (excitation at 320 nm) compared to the peptidyl-MCA. The amount of released MCA due to cleavage from ACE2 was calculated from the slope of the increase in fluorescence intensity and a standard curve with known MCA concentrations.

### 10. Virus Neutralization Assay

### Virus Strains

SARS-CoV-2-Munich-TUM-1 (EPI_ISL_582134) was isolated from a nasopharyngeal swab of a COVID-19 positive patient in Munich (January 2020) and grown and propagated on Vero E6 cells in DMEM medium (5% FCS, 1% penicillin/streptomycin, 200 mmol/L L-glutamine, 1% MEM-non-essential amino acids, 1% sodium-pyruvate (all from Gibco).

SARS-CoV-2 D614G was isolated from patient material in Munich, Germany (April 2020), grown on Caco-2 cells and propagated on Vero E6 cells.

SARS-CoV-Fra-1 from Frankfurt (AY291315.1) was grown and propagated on Vero E6 cells in DMEM medium (10% fetal calf serum (FCS), 100 μg/ml Streptomycin, 100 IU/ml Penicillin) (all from Gibco).

### Viral Neutralization Assay Followed by In-Cell ELISA

VeroE6 cells were plated in a 96-well plate at 1.6E04 cells/well in DMEM medium (Gibco) supplemented with 5% FCS, 1% penicillin-streptomycin, 200 mmol/L L-glutamine, 1% MEM-non-essential amino acids, 1% sodium-pyruvate (all from Gibco) and incubated overnight at 37° C. and 5% CO₂. Serial dilutions of the ACE2-Fc fusion proteins were mixed with virus in fresh media and pre-incubated for 1 hour at 37° C. The VeroE6 cells were infected at the MOI of 0.3 with the neutralized virus solution for 1 hour at 37° C. Next, the neutralization mix was removed, culture medium was added, and cells were incubated at 37° C. for 24 hours. Mock cells represent uninfected Vero E6 cells, incubated with culture medium. After 24 hours cells were washed once with PBS and fixed with 4% paraformaldehyde (ChemCruz) for 10 min at RT. Following a washing step with PBS, fixed VeroE6 cells were permeabilized with 0.5% saponin (Roth) in PBS for 10 min at room temperature. Next, the permeabilization solution was removed and cells were blocked with a mixture of 0.1% saponin and 10% goat serum (Sigma) in PBS with gentle shaking at RT for 1 hour. Subsequently, Vero E6 cells were incubated with a 1:500 dilution of an anti-dsRNA J2 antibody (Jena Bioscience) in PBS supplemented with 1% FCS at 4° C. overnight with shaking, followed by four washing steps with wash buffer (1×PBS supplemented with 0.05% Tween-20 (Roth)). Next, the plates were incubated with a 1:2000 dilution of a goat anti-mouse IgG2a-HRP antibody (Southern Biotech) in PBS supplemented with 1% FCS and incubated with gently shaking at RT for 1 hour. Following four washing steps, 3,3′,5,5′-Tetramethylbenzidin (TMB) substrate (Invitrogen) was added to the wells and incubated in the dark for 10 min. Colorimetric detection on a Tecan infinite F200 pro plate reader at 450 nm and at 560 nm was performed after stopping the color reaction by the addition of 2N H₂SO₄ (Roth). Following normalization against values obtained with uninfected Vero E6 cells, optical densities were transformed in percent neutralization values and half-maximal inhibitory concentrations (IC50 values) were calculated (Graphpad Prism).

### 11. Determination of Binding Affinity of the ACE2 Fusion Proteins to Fc-Receptors Using Surface Plasmon Resonance (SPR)

A Biacore T200 was used for the Fc-receptor binding studies. For the FcγRI and the FcγRIIIa experiments, the His-tagged FcγRI and FcγRIIIa with a concentration of 1.5 nM were captured by injection of the solutions for 90 seconds with a flow rate of 5 μL/min over a covalently immobilized anti-his tag antibody on a CM5 chip. The running buffer was HBS-EP+ pH 7.4 (Cytiva). Five different concentrations of the ACE2-Fc constructs were injected in a single-cycle kinetic mode (3.7-300 nM for the experiment with FcγRI and 25-2000 nM for FcγRIIIa). The FcγRI-binding data was fit to a heterogeneous ligand model and the first binding constant was reported. The FcγRIIIa-binding data was fit to a two-state reaction model to derive the binding constant. For the FcRn experiments, the FcRn was covalently immobilized on a CM5 chip to around 50 RU (Response Units). The sample buffer was HBS-EP+ pH 6.0 (Cytiva). The ACE2-Fc constructs were injected in five different concentrations from 205 to 8000 nM in a single-cycle kinetic mode. The FcRn binding was evaluated with a steady-state affinity fit.

### 12. Determination of Virus Neutralization with a Cell Viability Assay

### Virus Strains

SARS-CoV-2-Munich-TUM-1 (EPI_ISL_582134) was isolated from a nasopharyngeal swab of a COVID-19 positive patient in Munich (January 2020) and grown and propagated on Vero E6 cells in DMEM medium (5% FCS, 1% penicillin/streptomycin, 200 mmol/L L-glutamine, 1% MEM-non-essential amino acids, 1% sodium-pyruvate (all from Gibco).

SARS-CoV-2 D614G was isolated from patient material in Munich, Germany (April 2020), grown on Caco-2 cells and propagated on Vero E6 cells.

SARS-CoV-2 B.1.1.7 was obtained from Dr. Bugert from the Institute for Microbiology of the Bundeswehr (GISAID: EPI_ISL_755639) and propagated on Vero E6 cells.

SARS-CoV-2 B.1.351 was obtained from LGL (Oberschleißheim, Germany). It was isolated from a patient in Germany, grown on Caco-2 cells and propagated on Vero E6 cells. The identity of the variant was confirmed by sequencing.

24 h before infection, human lung epithelial A549 cells (ATCC-CCL-185), engineered to overexpress the human angiotensin-converting enzyme 2 receptor, ACE2 (A549-hACE2), were plated at 15,000 cells per well in a 96-well white well half area plate with clear bottom (Corning) in DMEM containing 2% fetal bovine serum, 100 U/mL penicillin-streptomycin and 1% NEAA.

SARS-CoV-2-Munich-TUM-1 and SARS-CoV-2 variants D614G, B1.1.7 and B.1.351 were grown on Vero E6 cells. For this, 15×10⁶ Vero E6 cells were seeded in each T150 flask one day before infection. The infection was performed by adding the virus with a MOI of 0.01 at 37° C., 5% CO₂. One hour after adding the virus the medium was changed to DMEM with 10% fetal bovine serum, 100 U/mL penicillin-streptomycin and 1% NEAA, 200 mmol/l L-glutamine and 1% sodium pyruvate (all from Gibco).

Infection was determined using a luminometric readout of virus-induced cytotoxicity. In brief, 72 h after infection cells were treated according to manufacturer's instructions: 15 μl CellTiter-Glo 2.0 reagent (Promega, Wisconsin, USA) were added to each well, incubated for 10 min in the dark at room temperature and luminescence was recorded (0.5 s integration time, no filter) using the Infinite F200 microplate reader (Tecan). Viability of cells and the corresponding infectious titer for each virus isolate was calculated by normalization of infected cells to untreated control cells (set to 100%). A serial dilution of constructs was performed and mixed with a defined volume of virus stocks of the indicated SARS-CoV-2 clinical isolates resulting in 80% cytotoxicity. After 1 h of pre-incubation, the mix of construct and the respective SARS-CoV-2 isolates was added to A549-hACE2 cells. 72 h after infection virus-induced cytotoxicity was determined as described above.

### 13. Determination of Virus Titer by a Plaque Assay

Viral titers were determined as described by Baer et al. (2014) J Vis Exp, e52065 with some modifications. Briefly, HepG2 or Vero E6 cells were plated in a 12-well plate at 5E05 cells/well in DMEM medium (Gibco) supplemented with 5% FCS, 1% P/S, 200 mmol/L L-glutamine, 1% MEM-NEAA, 1% sodium-pyruvate (all from Gibco) and incubated overnight at 37° C. and 5% CO₂. Cells were infected with serial dilution of virus sample in cell culture medium at 37° C. for one hour. After discarding the supernatant, 1 mL of 5% carboxymethylcellulose (Sigma) diluted in Minimum Essential Media (Gibco) was added per well and the plate was incubated at 37° C. until obvious plaques appeared. After removing the supernatant, cells were fixed with 10% paraformaldehyde (ChemCruz) at RT for 30 min. Next, a washing step with PBS was performed, followed by the addition of 1% crystal violet (Sigma; diluted in 20% methanol and water). Following an incubation time of 15 min at RT, the solution was washed away with PBS and the plate was dried. The viral titer (PFU/mL) of the sample was determined by counting the average number of plaques for a dilution and the inverse of the total dilution factor.

### B. Results

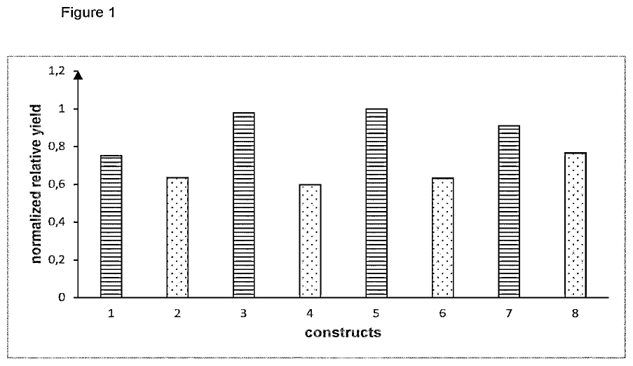

FIG. 1 shows that for those fusion proteins having the shorter ACE2 fragment comprising amino acids 18 to 732 (constructs 1, 3, 5 and 7) a higher yield compared to the fusion proteins comprising amino acids 18 to 740 of ACE2 (constructs 2, 4, 6 and 8) was obtained.

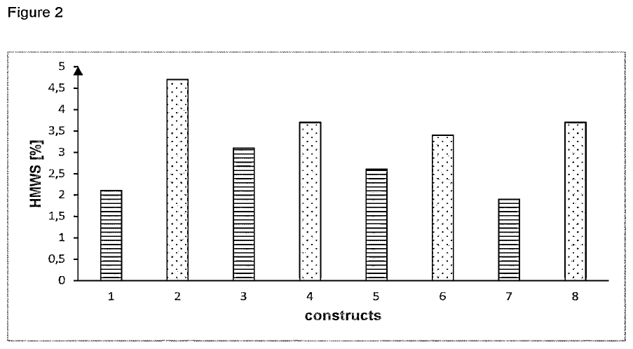

Further, the fusion proteins having the shorter ACE2 fragment comprising amino acids 18 to 732 (constructs 1, 3, 5 and 7) had a lower percentage of high molecular weight species indicating protein aggregates than the fusion proteins comprising amino acids 18 to 740 of ACE2 (constructs 2, 4, 6 and 8) (see FIG. 2). The same was observed after storage for three or six weeks (see FIG. 10).

In the surface plasmon resonance all constructs showed comparable binding to the Spike protein of SARS-CoV-2 (see Table 2):

TABLE 2

| Construct | Replicate | K_{D} (M) | kₒₙ (1/Ms) | k_{off} (1/s) |
|---|---|---|---|---|
| Construct 1 | 1 | 4.246E−9 | 9.352E+4 | 3.971E−4 |
| | 2 | 3.974E−9 | 9.417E+4 | 3.742E−4 |
| Construct 2 | 1 | 4.282E−9 | 9.958E+4 | 4.264E−4 |
| | 2 | 4.314E−9 | 1.039E+5 | 4.482E−4 |
| Construct 3 | 1 | 4.231E−9 | 9.006E+4 | 3.811E−4 |
| | 2 | 4.256E−9 | 9.314E+4 | 3.964E−4 |
| Construct 4 | 1 | 4.613E−9 | 9.213E+4 | 4.250E−4 |
| | 2 | 5.170E−9 | 8.718E+4 | 4.507E−4 |
| Construct 5 | 1 | 4.111E−9 | 9.585E+4 | 3.941E−4 |
| | 2 | 3.682E−9 | 9.523E+4 | 3.507E−4 |
| Construct 6 | 1 | 4.106E−9 | 9.451E+4 | 3.880E−4 |
| | 2 | 4.804E−9 | 9.659E+4 | 4.640E−4 |
| Construct 7 | 1 | 3.939E−9 | 9.114E+4 | 3.590E−4 |
| | 2 | 3.832E−9 | 9.087E+4 | 3.482E−4 |
| Construct 8 | 1 | 4.213E−9 | 9.224E+4 | 3.886E−4 |
| | 2 | 4.348E−9 | 1.090E+5 | 4.740E−4 |

Comparable results were obtained with the Spike protein of the delta variant of SARS-CoV2 (see Table 3):

TABLE 3

| Construct | K_{D} [M] | kₒₙ [1/Ms] | k_{off} [1/s] |
|---|---|---|---|
| Construct 1 | 1.44E−09 | 1.52E+05 | 2.17E−04 |
| Construct 5 | 1.44E−09 | 1.36E+05 | 1.94E−04 |

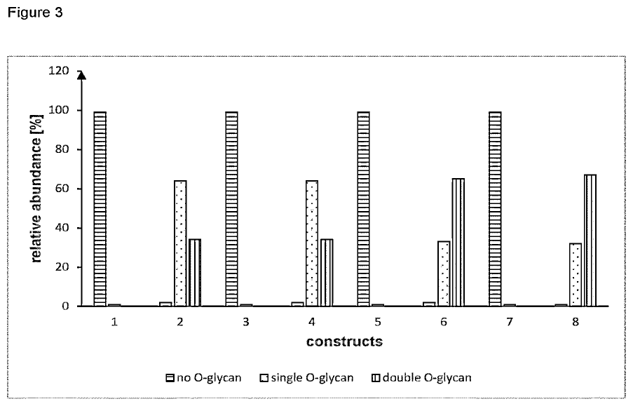

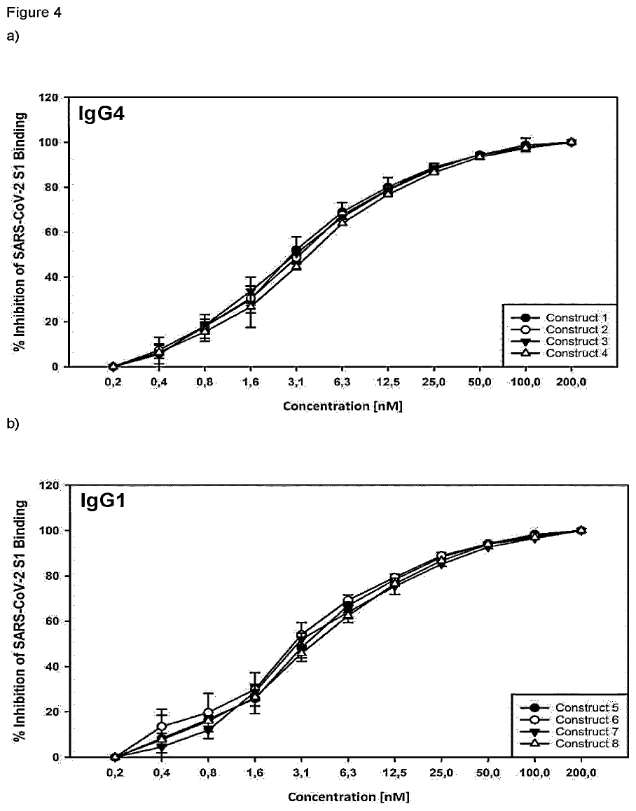

FIG. 3 shows that constructs 1, 3, 5 and 7 have essentially no O-glycosylation, while constructs 2, 4, 6, and 8 have varying amounts of single and double O-glycans.

FIG. 4 shows that all constructs 1 to 8 inhibited binding of SARS-CoV-2 Spike S1 protein to ACE2 as determined by ELISA 1.

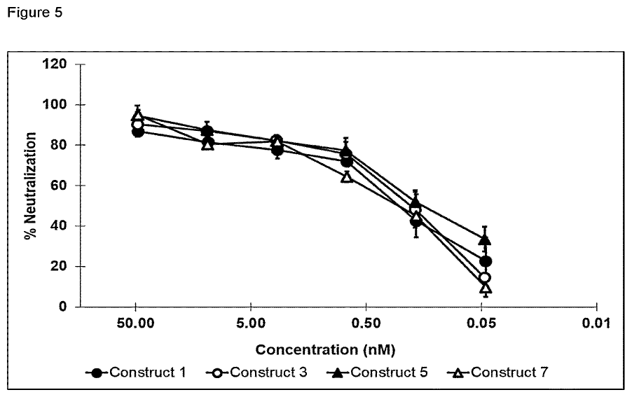

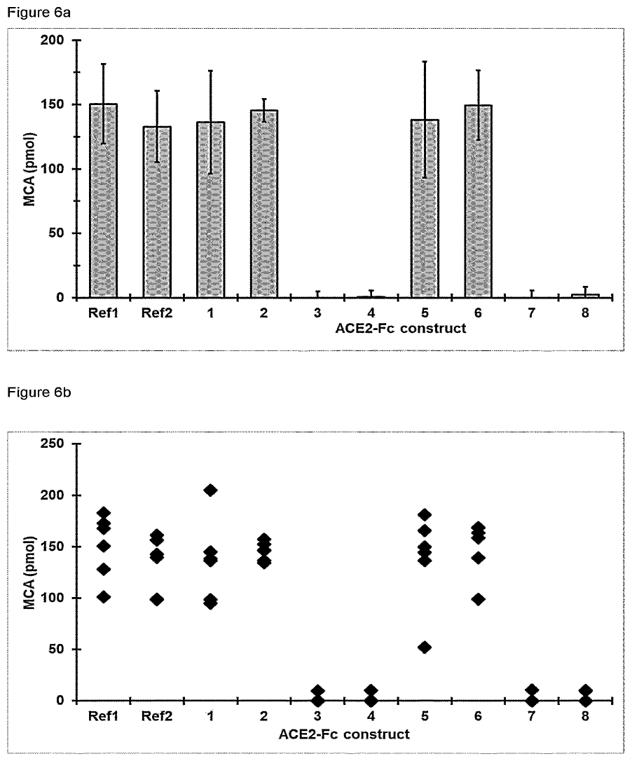

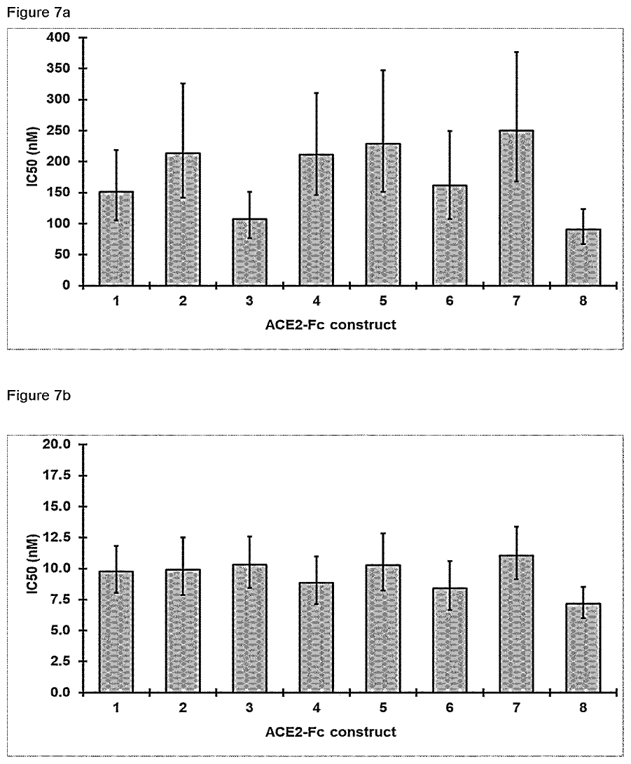

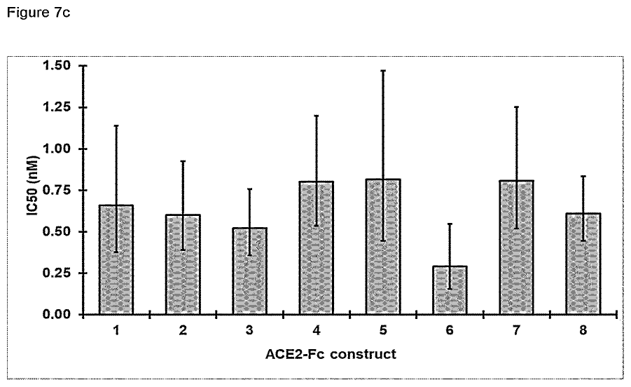

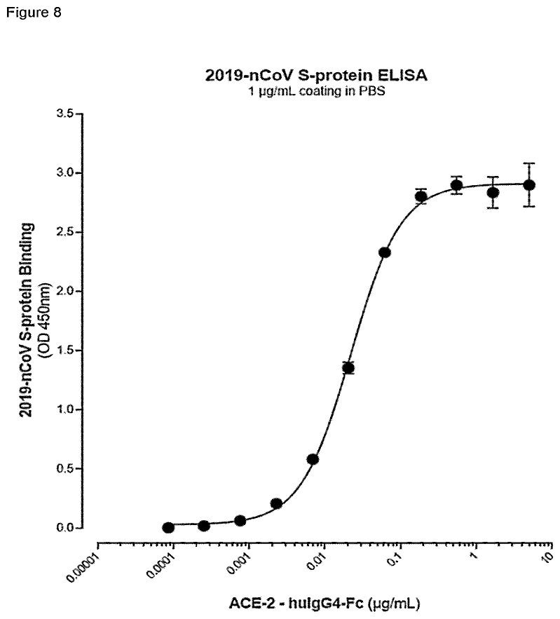

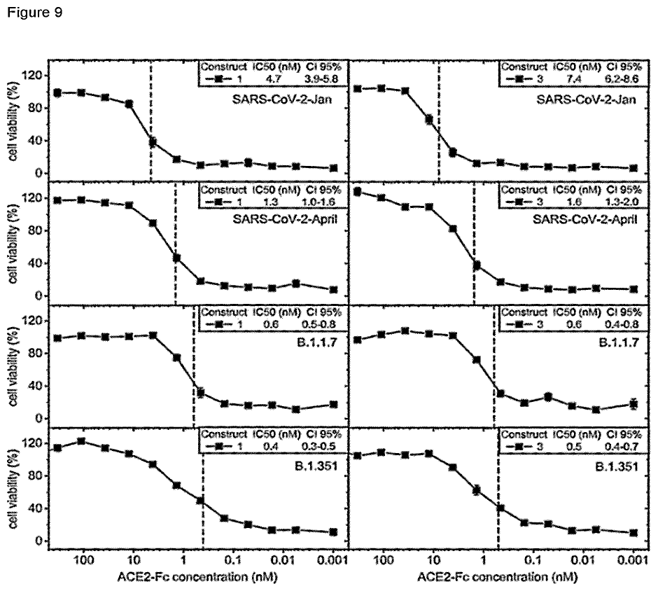

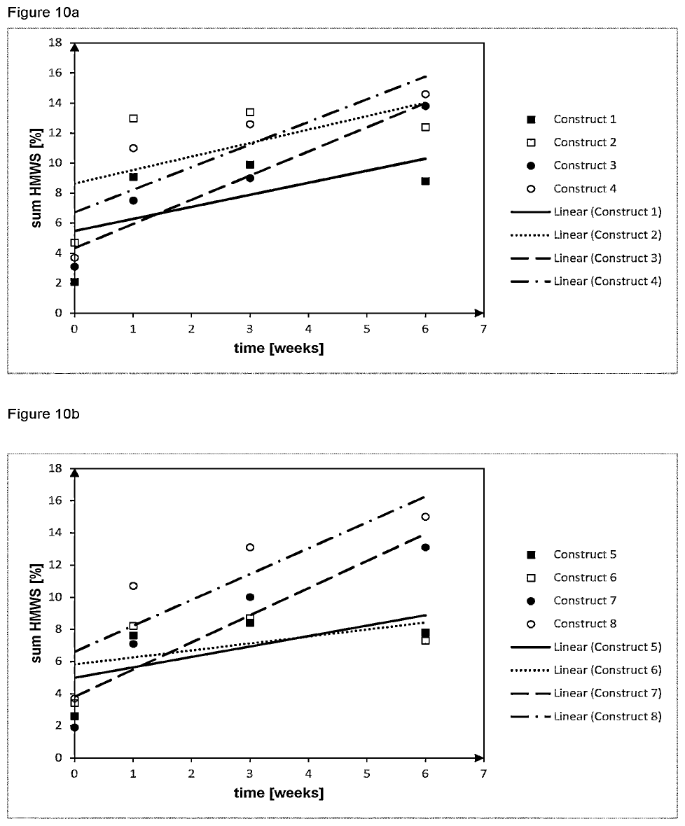

Constructs 1, 3, 5 and 7 almost completely inhibited infection of VeroE6 cells by SARS-CoV-2 strain Victoria/1/2020 (see FIG. 5). All constructs neutralized SARS-CoV-2 strain Victoria/1/2020 with IC50 values in the range of 0.5 nM.

Constructs 1, 2, 5 and 6 cleave the same amount of the synthetic peptidyl-MCA derivate after 30 min of incubation, while the constructs with mutations in the active ACE2 site have complete lost the enzymatic activity (see FIGS. 6a and 6b).

All constructs 1 to 8 neutralized SARS-CoV with IC50 values in the range of 150 nM (see FIG. 7a), SARS-CoV-2 with IC50 values in the range of 10 nM (see FIG. 7b) and SARS-CoV-2 D614G with IC50 values in the range of 1 nM (see FIG. 7c).

The ACE2-IgG4-Fc fusion proteins showed slightly lower affinity to FcγRI when compared to the IgG1 counterparts (see Table 4). The ACE2-IgG4-Fc showed no binding to FcγRIIIa, which contrasts with the ACE2-IgG1-Fc molecules (see Table 4). All four constructs had similar affinity to the FcRn (see Table 4).

TABLE 4
Binding affinities of ACE2-Fc constructs to Fc-receptors

| | FcγRI | FcγRIIIa | FcRn |
|---|---|---|---|
| Construct | K_{D} (nM) | K_{D} (μM) | K_{D} (μM) |
| 1 | 45 ± 0.2 | No binding | 2.9 ± 0.68 |
| 3 | 43 ± 5.2 | No binding | 3.9 ± 0.62 |
| 5 | 10 ± 2.2 | 0.6 ± 0.45 | 3.4 ± 0.05 |
| 7 | 11 ± 0.8 | 0.8 ± 0.54 | 3.5 ± 0.62 |

Mean ± SD of duplicate measurements

Construct 1 binds to the Spike protein of wild-type SARS-CoV-2 with an EC50 value of 25.9 ng/ml as determined by ELISA 2 (see FIG. 8).

FIG. 9 shows that the ACE2-Fc fusion proteins according to SEQ ID Nos. 6 and 8 (constructs 1 and 3) neutralize all clinical isolates of SARS-CoV-2 tested. The more infectious the SARS-CoV-2 variant is, the better the fusion proteins neutralize it, as evidenced by the lower IC50 (50% inhibitory concentration) values. In particular, the fusion proteins are most effective against the SARS-CoV-2 variants B.1.1.7 and B.1.351 which have been shown to be refractory to neutralization by most monoclonal antibodies directed against the N-terminal domain of the Spike protein and monoclonal antibodies against the receptor-binding motif (Wang et al. (2021) Nature). In addition, the B.1.351 variant shows an escape from neutralization by convalescent plasma and sera from individuals who have been vaccinated.

The IC50 values for the different constructs and clinical isolates are shown in Table 5.

TABLE 5
IC50 and 95% confidence interval for different constructs and
clinical isolates

| | | IC50 | |
|---|---|---|---|
| Construct | Isolate | (nM) | CI95 |
| 1 | SARS-COV-2-Munich-TUM-1 | 4.7 | 3.9-5.8 |
| 1 | SARS-COV-2 D14G | 1.3 | 1.0-1.6 |
| 1 | SARS-COV-2 B.1.1.7 | 0.6 | 0.5-0.8 |
| 1 | SARS-COV-2 B.1.351 | 0.4 | 0.3-0.5 |
| 3 | SARS-COV-2-Munich-TUM-1 | 7.4 | 6.2-8.6 |
| 3 | SARS-COV-2 D14G | 1.6 | 1.3-2.0 |
| 3 | SARS-COV-2 B.1.1.7 | 0.6 | 0.4-0.8 |
| 3 | SARS-COV-2 B.1.351 | 0.5 | 0.4-0.7 |

1. A fusion protein comprising a first part comprising a fragment of human ACE2 or a variant of said fragment, said human ACE2 having the amino acid sequence according to SEQ ID No. 1, and a second part comprising a variant of the Fc portion of human IgG1, said variant having the amino acid sequence according to SEQ ID No. 19.

2. The fusion protein according to claim 1,
wherein the first part and the second part are linked by the amino acid sequence according to SEQ ID No. 15.

3. The fusion protein according to claim 1,
wherein the fragment of human ACE2 consists of a protein with the amino acid sequence according to SEQ ID No. 2.

4. The fusion protein according to claim 1,
wherein the fragment of human ACE2 is the extracellular domain of ACE2 consisting of a protein with the amino acid sequence according to SEQ ID No. 3.

5. The fusion protein according to claim 1,
having the amino acid sequence according to SEQ ID No. 20 or 21.

6. The fusion protein according to claim 1, wherein the variant of the human ACE2 fragment is an enzymatically inactive variant of human ACE2.

7 The fusion protein according to claim 6,
wherein the enzymatically inactive variant of human ACE2 comprises a H374N and a H378N mutation, the numbering referring to SEQ ID No. 1.

8-9. (canceled)

10. The fusion protein according to claim 6,
wherein the enzymatically inactive variant of human ACE2 comprises a mutation at position 273, the numbering referring to SEQ ID No. 1.

11-13. (canceled)

14. The fusion protein according to claim 1, wherein the variant of the human ACE2 fragment comprises a S645C mutation, the numbering referring to SEQ ID No. 1.

15-82. (canceled)

83. A nucleic acid molecule comprising a nucleic acid sequence encoding the fusion protein according to claim 1.

84. An expression vector comprising the nucleic acid molecule according to claim 83.

85. A host cell comprising (i) the nucleic acid molecule according to claim 83 or (ii) an expression vector comprising the nucleic acid molecule.

86. A method for producing the fusion protein according to claim 1, comprising culturing a host cell in a suitable culture medium, wherein the host cell comprises (i) a nucleic acid molecule comprising a nucleic acid sequence encoding the fusion protein or (ii) an expression vector comprising a nucleic acid molecule comprising a nucleic acid sequence encoding the fusion protein.

87. (canceled)

88. A method for preventing and/or treating an infection with a coronavirus binding to ACE2, comprising administering the fusion protein according to claim 1.

89. The method according to claim 88, wherein the coronavirus binding to ACE2 is selected from the group consisting of SARS, SARS-COV-2 and NL63.

90. The method Fusion protein for use according to claim 88, wherein the fusion protein is administered in combination with an anti-viral agent.

91. (canceled)

92. The method according to claim 88, wherein the fusion protein is administered in combination with an antibody.

93. (canceled)

94. A method for treating hypertension (including high blood pressure), congestive heart failure, chronic heart failure, acute heart failure, contractile heart failure, myocardial infarction, arteriosclerosis, kidney failure, renal failure, Acute Respiratory Distress Syndrome (ARDS), Acute Lung Injury (ALI), chronic obstructive pulmonary disease (COPD), pulmonary hypertension, renal fibrosis, chronic renal failure, acute renal failure, acute kidney injury, inflammatory bowel disease and multi-organ dysfunction syndrome, comprising administering the fusion protein of claim 1.

95. A pharmaceutical composition comprising an effective amount of the fusion protein according claim 1 and a pharmaceutically acceptable carrier or excipient.

96. A pharmaceutical composition according to claim 95, further comprising an anti-viral agent.
